(19) United States
(12) Patent Application Publication
(10) Pub. No.: US 2024/0018611 A1
(43) Pub. Date: Jan. 18, 2024

(54) IMMUNE MOLECULE VIRUS PARTICLE DETECTION KIT

(71) Applicant: Chongqing Medical University, Chongqing (CN)

(72) Inventors: Deqiang Wang, Chongqing (CN); Yuanyan Ma, Chongqing (CN); Xiaoli Wu, Chongqing (CN); Junye Liu, Chongqing (CN); Xuefei Cai, Chongqing (CN); Shaocheng Zhang, Chongqing (CN); Wen Wang, Chongqing (CN); Shenglan Mao, Chongqing (CN); Shimei Shen, Chongqing (CN); Yalan Li, Chongqing (CN); Jinyu Huang, Chongqing (CN); Zixin Wen, Chongqing (CN); Yunyou Wang, Chongqing (CN)

(21) Appl. No.: 18/475,264

(22) Filed: Sep. 27, 2023

Related U.S. Application Data
(63) Continuation of application No. PCT/CN2022/080846, filed on Mar. 15, 2022.

(30) Foreign Application Priority Data
Jun. 2, 2021 (CN) 202111212401X
Jun. 2, 2021 (CN) 2021106138706
Jun. 2, 2021 (CN) 2022100273107

(51) Int. Cl. C12Q 1/70; C12Q 1/6851

(52) U.S. Cl. CPC C12Q 1/701; C12Q 1/6851; C12Q 1/6804

(57) ABSTRACT

An immune molecule virus particle detection kit is provided. A monoclonal antibody of a virus envelope antigen is modified with biotin, magnetic beads are coupled with streptavidin, and the biotin-modified monoclonal antibody is incubated with a virus-containing solution to form a complex with virus particles or antigens, and then the streptavidin-coupled magnetic beads are added for incubation. The streptavidin on the magnetic beads binds with the biotin-modified monoclonal antibody with high specificity and affinity, and then specifically captures the virus particles with envelopes. After separating a supernatant through a magnetic separator, complete viral particles, empty-shell viruses, and free envelope antigens can be separated from other virus components, and the magnetic bead conjugates can be qualitatively or quantitatively detected through polymerase chain reaction (PCR) amplification. The immune molecule virus particle detection method using the kit has the characteristics of simplicity, rapidity, accuracy, and low cost, and has good application prospects.

### CROSS-REFERENCING OF RELATED APPLICATIONS

This application claims the priority of a Chinese patent application No. CN202110613870.6 filed on Jun. 2, 2021, a Chinese patent application No. CN202111212401.X filed on Oct. 18, 2021, and a Chinese patent application No. CN202210027310.7 filed on Jan. 11, 2022. The entire contents of the above-mentioned applications are hereby incorporated by reference.

### TECHNICAL FIELD

The disclosure relates to the technical field of molecular biology and relates to virus particle detection, and more particularly to an immune molecule virus particle detection kit.

### BACKGROUND

Viral infection is a process in which viruses invade the body through one or more ways and proliferate in susceptible host cells. The essence of viral infection is the process of interaction between the viruses and the body, the viruses and the susceptible host cells. Viral infection often causes different degrees of damage or viral diseases due to different types of viruses and health status. Viral pathogenesis begins with invasion of the host and infection of cells, in which the binding of a receptor-binding protein located in a viral envelope to a host receptor is the development of infection, and a viral genome (ribonucleic acid abbreviated as RNA or deoxyribonucleic acid abbreviated as DNA) located in a core of the virus is the basis of viral transcription and replication. If the virus has the activity of infecting the host, its basic feature is that the receptor-binding protein located in the envelope is active, and the virus genome at the core of the virus particle remains complete.

The existing virus detection methods are mainly immunoassay and molecular detection. Taking the detection of novel coronavirus as an example, the current immunoassay of novel coronavirus is mainly based on antigen detection of nucleocapsid protein (also referred to as N protein) of novel coronavirus, antibody detection based on the N protein and/or spike protein (also referred to as S protein) (or receptor-binding domain abbreviated as RBD) immunization, and nucleic acid detection based on viral RNA. The existing commercial or literature-reported virus immunoassays and molecular detections only detect viral antigens or nucleic acids, and cannot detect virus particles with infectious activity.

### SUMMARY

In order to solve the problems in the related art, according to a first aspect of the disclosure, the disclosure provides an immune molecule virus particle detection kit.

To achieve the above objectives, technical solutions of the disclosure are as follows.

Specifically, an immune molecule virus particle detection kit includes a monoclonal antibody, biotin, magnetic beads and streptavidin. The monoclonal antibody is a monoclonal antibody of a virus envelope antigen. The process of using the kit to detect virus particles is as follows. The monoclonal antibody of virus envelope antigen is modified with biotin, and the magnetic beads are coupled with streptavidin. The biotin-modified monoclonal antibody is incubated with a virus-containing solution to thereby form a complex with the virus particles or the virus envelope antigen, and then the magnetic beads coupled with the streptavidin is added for incubation. The streptavidin coupled on the magnetic beads is bound to the biotin-coupled monoclonal antibody, thereby capturing the virus particles with envelopes. After separating supernatant through a magnetic separator, complete virus particles, empty-shell viruses, and free envelope antigens can be separated from other virus components to thereby obtain magnetic bead conjugates, and then the magnetic bead conjugates can be qualitatively or quantitatively detected through polymerase chain reaction (PCR), fluorescence quantitative PCR, or digital PCR, and isothermal amplification, etc.

In an embodiment, an immune molecule virus particle detection kit is provided. A monoclonal antibody of a virus envelope antigen (a host receptor-binding virus protein) is coupled with magnetic beads, and then the magnetic beads coupled with the monoclonal antibody are incubated with a virus-containing solution to specifically capture virus particles with envelopes. After separating supernatant through a magnetic separator, complete virus particles, empty-shell viruses, and free envelope antigens can be separated from other virus components (a protein-virus RNA/DNA complex, a free virus gene fragment, etc.) to thereby obtain magnetic bead conjugates, and then the magnetic bead conjugates can be qualitatively or quantitatively detected through polymerase chain reaction (PCR), fluorescence quantitative PCR, or digital PCR, and isothermal amplification, etc. (sub-virus particles such as empty-shell viruses cannot be amplified due to the lack of a genome, and only complete virus particles have positive molecular detection signals). Therefore, the virus genes detected by nucleic acid amplification are those from complete viruses, and the detection signal is the signal of complete virus particles.

The virus envelope antigen is a host receptor-binding viral protein. The other virus components are sub-virus particle components, such as a protein-virus RNA/DNA complex, a free virus gene fragment, etc. The PCR amplification includes fluorescence quantitative PCR, digital PCR, recombinase polymerase amplification (RPA), enzymatic recombinase amplification (ERA), loop-mediated isothermal amplification (LAMP), etc.

The biotin-modified monoclonal antibody is obtained by dialyzing the monoclonal antibody with a sodium bicarbonate buffer with a power of hydrogen (pH) value of 8.0 or a boric acid buffer with a value of pH 8.6 to obtain a monoclonal antibody solution, adding biotin dissolved in dimethyl sulfoxide (DMSO) into the monoclonal antibody solution, continuously stirring at room temperature, and keeping the temperature for 2-4 hours (h); adding ammonium chloride (NH₄Cl), and stirring at room temperature for 5-15 minutes (min); removing free biotin to obtain a loading sample; loading the loading sample on a molecular sieve column, eluting with phosphate buffered saline (PBS), and collecting proteins; adding sodium azide and bovine serum albumin (BSA) to form the biotin-modified monoclonal antibody as a product to be combined.

The streptavidin-coupled magnetic beads is obtained by taking the magnetic beads into an Eppendorf (EP) tube, performing magnetic separation on the magnetic beads, and washing with a precooled 4-morpholinoethanesulfonic acid (MES) buffer; applying a magnetic field to remove a supernatant, adding an N-hydroxysuccinimide (NHS) solution and a 1-Ethyl-3-[3-dimethylaminopropyl]carbodiimide hydrochloride (EDC) solution with a same amount as the NHS solution into the EP tube, shaking, and activating the magnetic beads at 20-30 Celsius degree (° C.) for 20-40 min; washing the activated magnetic beads with the precooled MES buffer with a magnetic rack; diluting streptavidin to be coupled with the precooled MES buffer to obtain a diluted streptavidin suspension, resuspending the activated magnetic beads after washing with the precooled MES buffer, and shaking to disperse all the magnetic beads, thereby to obtain an activated magnetic bead suspension; taking and adding the activated magnetic bead suspension into the diluted streptavidin suspension, and rotating and mixing uniformly at 4° C. for 4 h; applying the magnetic field to removing a supernatant, adding a BSA blocking solution into the EP tube, and rotating at 20-30° C. for 20-40 min; washing the magnetic beads after coupling by the magnetic rack with the PBS; transferring a preservation solution into the EP tube, suspending the magnetic beads coupled with the streptavidin, and preserving at 4° C., so as to obtain the streptavidin-coupled magnetic beads.

The virus particles bound to the biotin-modified monoclonal antibody and captured by the streptavidin-coupled magnetic beads are performed by: taking a cell supernatant into another EP tube, adding the biotin-modified monoclonal antibody for incubation and binding, and rotating at 20-30° C. for 5-15 min; adding the streptavidin-coupled magnetic beads, uniformly mixing, and rotating and binding at 20-30° C. for 30-50 min; and discarding the supernatant after magnetic field is applied to obtain the complete virus particles, the empty-shell viruses and the free envelope antigens.

In an embodiment of the disclosure, the virus particles are complete virus particles (i.e., virus particles with infectious activity) selected from the group consisting of hepatitis A virus (HAV), hepatitis B virus (HBV), hepatitis C virus (HCV), hepatitis D virus (HDV), hepatitis E virus (HEV), novel coronavirus (SARS-CoV-2), human immunodeficiency virus (HIV), influenza virus, Partial pulmonary virus, human papillomavirus (HPV), herpes virus, herpesvirus hominis, Zika virus, Ebola virus (EBV), human T-lymphocytic virus, avian influenza virus, hog cholera virus (CSFV), poliovirus, rabies virus, adenovirus, and lentivirus.

The disclosure provides the immune molecule virus particle detection kit. The monoclonal antibody of the virus envelope antigen is modified with the biotin, the magnetic beads are coupled with the streptavidin, the biotin-modified monoclonal antibody is incubated with a virus-containing solution to form the complex with virus particles or antigens, and then the magnetic beads coupled with the streptavidin are added for incubation. The streptavidin on the magnetic beads binds with the biotin-modified antibody with high specificity and affinity, and then specifically captures the virus particles with the envelopes. After separating the supernatant through the magnetic separator, the complete virus particles, the empty-shell viruses, and the free envelope antigens can be separated from other virus components (protein-virus RNA/DNA complexes, free virus gene fragments, and other sub-virus particle components), and then the magnetic bead conjugates can be qualitatively or quantitatively detected through PCR amplification. The sub-virus particles such as the empty-shell viruses cannot be amplified due to the lack of genome, and only complete virus particles have positive molecular detection signals. Therefore, the virus genes detected by PCR (or fluorescence quantitative PCR, digital PCR), isothermal amplification, etc. are the virus genes from the complete viruses, and the detection signal is the signal of the complete virus particles.

In a second aspect of the disclosure, the disclosure provides a method for detecting immune capture molecules of complete HBV virus particles (complete virus particles, core particles, etc.). Using carboxy magnetic beads as a medium, specific antibodies are coupled with the carboxy magnetic beads, or streptavidin-magnetic beads-biotin-modified antibodies are used as a medium for virus particle capture and separation, followed by nucleic acid amplification detection.

Specifically, a method for detecting immune capture molecules of complete HBV virus particles includes antibody-magnetic bead coupling, HBV virus particle capture and real-time fluorescence quantitative PCR. The antibody-magnetic bead coupling includes: mixing carboxy magnetic beads, an NHS solution and an EDC with a same amount as the NHS solution in a buffer to activate magnetic beads, and mixing and reacting the activated magnetic beads with an antibody to be coupled in a coupling buffer to obtain an antibody-magnetic bead coupling reaction product. The antibody to be coupled is at least one of a PreS1 antibody and an HBc antibody.

The antibody-magnetic bead coupling specifically includes: taking the carboxy magnetic beads into an EP tube, performing magnetic separation on the carboxy magnetic beads, and washing with an MES buffer for three times; applying a magnetic field to remove a supernatant, quickly adding the NHS solution and the EDC solution with the same amount of the NHS solution into the EP tube, shaking vigorously, and continuously activating the carboxy magnetic beads at 25° C. for 30 min; washing the activated carboxy magnetic beads with the MES buffer with a magnetic rack for three to five times; diluting the antibody to be coupled with the MES buffer until a final antibody concentration is 0.1 grams per liter (g/L) to 1.5 g/L to obtain a diluted antibody suspension; resuspending the activated carboxy magnetic beads with the MES buffer and shaking vigorously to ensure that the activated carboxy magnetic beads are completely dispersed to thereby obtain an activated magnetic bead suspension; taking and adding the activated magnetic bead suspension into the diluted antibody suspension for 3-8 times, mixing immediately after adding the activated magnetic bead suspension each time, rotating and mixing evenly at 4° C. for 4 h; preparing 3-6% of BSA blocking solution (0.1 M MES, pH 5.0-8.0); applying the magnetic field to remove a supernatant, quickly adding the BSA blocking solution into the EP tube, and rotating at 25° C. for 10-40 min; washing the carboxy magnetic beads after coupling by the magnetic rack with PBS for three times; transferring a preservation solution to the EP tube, suspending the carboxy magnetic beads, and preserving at 4° C.

A cell supernatant containing HBV virus particles or a serum of hepatitis B virus infected persons (5-50 μL) is captured by the antibody-coupled magnetic bead preservation solution, the supernatant is separated with a magnetic force, and washed twice with PBS. The cell supernatant or hepatitis B patient serum is taken into an EP tube, and diluted with PBS. The antibody-magnetic bead conjugates are added to the diluted sample, mixed evenly, rotated at 25° C. for 20-40 min for virus particle capture (complex).

In an embodiment, the streptavidin-coupled magnetic beads can be used. The streptavidin-coupled magnetic beads is obtained by taking the magnetic beads into an EP tube, performing magnetic separation on the magnetic beads, and washing with a precooled MES buffer; applying a magnetic field to remove a supernatant, adding an NHS solution and a EDC solution with a same amount as the NHS solution into the EP tube, shaking, and activating the magnetic beads at 20-30° C. for 20-40 min; washing the activated magnetic beads with the precooled MES buffer with a magnetic rack; diluting streptavidin to be coupled with the precooled MES buffer to obtain a diluted streptavidin suspension, resuspending the activated magnetic beads after washing with the precooled MES buffer, and shaking to disperse all the magnetic beads, thereby to obtain an activated magnetic bead suspension; taking and adding the activated magnetic bead suspension into the diluted streptavidin suspension, and rotating and mixing uniformly at 4° C. for 4 h; applying the magnetic field to removing a supernatant, adding a BSA blocking solution into the EP tube, and rotating at 20-30° C. for 20-40 min; washing the magnetic beads after coupling by the magnetic rack with the PBS; transferring a preservation solution into the EP tube, suspending the magnetic beads coupled with the streptavidin, and preserving at 4° C., so as to obtain the streptavidin-coupled magnetic beads.

The biotin-modified monoclonal antibody is obtained by dialyzing the monoclonal antibody with a sodium bicarbonate buffer with a pH value of 8.0 or a boric acid buffer with a value of pH 8.6 to obtain a monoclonal antibody solution, adding biotin dissolved in DMSO into the monoclonal antibody solution, continuously stifling at room temperature, and keeping the temperature for 2-4 h; adding NH₄Cl, and stifling at room temperature for 5-15 min; removing free biotin to obtain a loading sample; loading the loading sample on a molecular sieve column, eluting with PBS, and collecting proteins; adding sodium azide and BSA to form the biotin-modified monoclonal antibody as a product to be combined.

The cell supernatant containing HBV virus particles or the serum of hepatitis B infected patients (5-50 μL) is incubated with PreS1 or HBc monoclonal antibodies that have been modified with biotin (24° C., 10-30 min), then the streptavidin-coupled magnetic beads are added for further incubation and preservation (24° C., 10-30 min). The supernatant is separated with the magnetic force and washed with PBS twice. The cell supernatant or the serum of hepatitis B infected patients are taken into the EP tube and diluted with PBS, and the antibody-magnetic bead conjugates are added to the diluted sample, mixed evenly, rotated at 25° C. for 20-40 min for virus particle capture (complex).

In an embodiment of the disclosure, the real-time fluorescence quantitative PCR is performed according to the following procedures: uracil N-glycosylase (UNG) reaction at 50° C. for 2 min, one cycle; Taq enzyme activation at 94° C. for 5 min, one cycle; denaturation at 94° C. for 15 seconds (s), 45 cycles; annealing, extension and fluorescence collection at 57° C. for 30 s, 45 cycles; and instrument cooling at 25° C. for 10 s, one cycle.

The real-time fluorescence quantitative PCR mentioned above is: resuspending the captured complex with 50 microliters (μL) PBS to obtain a captured complex suspension, transferring the captured complex suspension to 8-strip PCR tubes, and removing a supernatant of the 8-strip PCR tubes with the magnetic rack of a PCR plate; placing required reagents at room temperature in advance to avoid light, setting standards A-D, a negative control and a positive control, adding 5 μL sample release agent to each well of the 8-strip PCR tubes, instantly centrifuging, beating and mixing evenly, and standing in the dark for 10 min; preparing a PCR mixed solution per person comprising 38 μL reaction solution+2 μL enzyme mixed solution+0.2 μL internal standard; and performing the real-time fluorescence quantitative PCR for cyclic amplification detection according to the following procedures: UNG reaction at 50° C. for 2 min, one cycle; Taq enzyme activation at 94° C. for 5 min, one cycle; denaturation at 94° C. for 15 s, 45 cycles; annealing, extension and fluorescence collection at 57° C. for 30 s, 45 cycles; and instrument cooling at 25° C. for 10 s, one cycle.

The disclosure provides the method for detecting immune capture molecules of complete HBV virus particles (including complete virus particles, core particles, etc.), using the carboxy magnetic beads as the medium to couple the specific antibodies, or using the streptavidin magnetic beads-biotin-modified antibodies as the medium to capture and separate virus particles, and then perform nucleic acid amplification detection.

The experiment shows that the method of the disclosure can successfully capture and separate the virus particles in the sample, and can distinguish different components of the virus particles in the sample due to different magnetic beads coupled antibodies. In addition, with the increase of sample size and antibody-magnetic bead complexes, the effect of virus enrichment can be achieved. Surprisingly, through the capture method of the disclosure, the composition of the virus particles in the cell supernatant and serum are different, and NCs particles account for a large proportion in the cell supernatant, while HBV Dane particles are mainly in the serum, especially in the case of high titer serum load. The high proportion of NCs particles in the cell supernatant may explain the low infectivity of the virus collected from the cell supernatant. The low content of NCs particles in serum may be due to the presence of strong and persistent anti-core antibodies in the blood circulation of most HBV infected patients, resulting in the rapid clearance of high immunogenicity naked capsids. Moreover, as the copies of HBV DNA in the serum increases, the content of complete virus particles in the serum shows a significant increase trend, indicating that the detection of complete virus particles may serve as a new serum marker. The disclosure has important practical value and is worth vigorously promoting in clinical practice.

In a third aspect of the disclosure, the disclosure provides a novel coronavirus (SARS-CoV-2) pseudovirus system containing both an envelopeprotein (spike) and a virus gene sequence.

Specifically, a pseudovirus system is provided. Lentivirus expression plasmids are integrated with a segmental genome of SARS-CoV-2, and a SARS-CoV-2 spike (S) glycoprotein is expressed on an envelope to simulate a functional structure of the SARS-CoV-2. The segmental genome of the SARS-CoV-2 is a sequence containing an ORF1ab (15415-15540), an N gene (28750-29150), and an E gene (26360-26381) of the SARS-CoV-2. The lentivirus expression plasmids are pCMV3-2019-nCoV-Spike (S1+S2), pLV-SARS-CoV-2-N-GFP, and pMD2 plasmids.

The pseudovirus system is produced by co-transfecting HEK-293FT cells with the pCMV3-2019-nCoV-Spike(S1+S2) plasmid, the pLV-SARS-CoV-2-N-GFP plasmid and the pMD2 plasmid by Lipofectamine™ 8000, and collecting a virus supernatant after co-transfection and mixing, centrifuging the virus supernatant at 4° C. at 3,000 g for 15 min to remove cell debris, placing a cell supernatant after the centrifuging on a 20% sucrose solution and obtaining a virus precipitate by centrifugation with a Beckman SW28 rotor at 4° C. at 125,000 rpm (112,000 g) for 15 h. The pseudovirus can infect cells once and does not have the self-replicate ability, resulting in high biological safety.

In a fourth aspect of the disclosure, the disclosure provides a method for detecting complete virus particles of novel coronavirus (SARS-CoV-2).

Specifically, a method for detecting complete virus particles of novel coronavirus (SARS-CoV-2) includes pseudovirus generation, pseudovirus identification, affinity antibody screening, carboxy magnetic beads and antibody coupling, SARS-CoV-2 quantitative RT-qPCR detection. The pseudovirus is generated by co-transfecting HEK-293FT cells with a pCMV3-2019-nCoV-Spike(S1+S2) plasmid, a pLV-SARS-CoV-2-N-GFP plasmid and a pMD2 plasmid by Lipofectamine™ 8000, collecting a virus supernatant after co-transfection and mixing, centrifuging the virus supernatant to remove cell debris, placing a cell supernatant after the centrifuging on a 20% sucrose solution, and obtaining a virus precipitate by centrifugation with a Beckman SW28 rotor.

The pseudovirus is generated by co-transfecting HEK-293FT cells with a pCMV3-2019-nCoV-Spike(S1+S2) plasmid, a pLV-SARS-CoV-2-N-GFP plasmid and a pMD2 plasmid by Lipofectamine™ 8000, collecting a virus supernatant at 48 h and 72 h after co-transfection and mixing, centrifuging the virus supernatant at 3000 g at 4° C. for 10 min to remove the cell debris, placing a cell supernatant on 20% sucrose solution, and obtaining the virus precipitate containing a SARS-CoV-2 pseudovirus by centrifugation with a Beckman SW28 rotor at 4° C. 25,000 rpm (112,000 g).

The pseudovirus identification involves performing pseudovirus infection in vitro by transfecting HEK-293FT cells overexpressing human angiotensin-converting enzyme 2 (hACE2) or transduced with empty lentivirus plasmid with the SARS-CoV-2 pseudovirus and a control pseudovirus encoding green fluorescent protein (GFP) into a 48-well plate for 48 h and 72 h, observing the pseudovirus infection under a fluorescence microscope and collecting a supernatant at 72 h, and detecting secretion of virus particles by a fluorescence quantitative PCR; determining efficiency of S protein fusion into the SARS-CoV-2 pseudovirus by western blotting with mouse monoclonal antibody anti-SARS-CoV-2 S (S2), and using a 2019-nCoV nucleic acid detection kit (Sansure Biotech Inc., China) to detect an ORF1ab gene of the SARS-CoV-2 in the SARS-CoV-2 pseudovirus by RT-qPCR to ensure successful integration of a virus genome of the SARS-CoV-2 into a lentivirus.

The affinity antibody screening of the disclosure is: performing SDS-PAGE and agarose gel electrophoresis respectively on a SARS-CoV-2 pseudovirus lysate and virus particles for resolution and membrane transfer, using mouse/human anti-SARS-CoV-2 S/M monoclonal antibodies as primary antibodies and horseradish peroxidase (HRP)-sheep anti-mouse monoclonal antibodies as secondary antibodies to screen an antibody with optimal specificity and affinity.

The carboxy magnetic beads and antibody coupling is: activating the carboxy magnetic beads continuously with an NHS solution and an EDC solution at 25° C. for 30 min to obtain activated carboxy magnetic beads MSP-COOH-F1; adding the activated carboxy magnetic beads MSP-COOH-F1 to a diluted antibody CQ25, mixing and rotating at 4° C. for 4 h, separating a supernatant to obtain an antibody-coupled magnetic bead complex, blocking the antibody-coupled magnetic bead complex with 1% BSA solution at 25° C. for 30 min; evaluating a coupling effect by the SDS-PAGE and Coomassie blue staining with the antibody-coupled magnetic bead complex and the separated supernatant.

The SARS-CoV-2 quantitative RT-qPCR detection described in the disclosure involves mixing the antibody-coupled magnetic bead complex and the SARS-CoV-2 pseudovirus in a PBS buffer at room temperature for 45 min to obtain a captured complex, and detecting a SARS-CoV-2 RNA level of the captured complex by using a novel coronavirus nucleic acid detection kit in a Bio-Rad CFX96 system.

In an embodiment, a method for detecting complete virus particles of SARS-CoV-2 includes pseudovirus generation, pseudovirus identification, affinity antibody screening, carboxy magnetic beads and antibody coupling, and SARS-CoV-2 nucleic acid amplification detection (quantitative RT-qPCR or isothermal amplification). The pseudovirus is generated by co-transfecting HEK-293FT cells with a pCMV3-2019-nCoV-Spike(S1+S2) plasmid, a pLV-SARS-CoV-2-N-GFP plasmid and a pMD2 plasmid by Lipofectamine™ 8000, collecting a virus supernatant at 48 h and 72 h after co-transfection and mixing, centrifuging the virus supernatant at 3000 g at 4° C. for 10 min to remove the cell debris, placing a cell supernatant on 20% sucrose solution, and obtaining the virus precipitate containing a SARS-CoV-2 pseudovirus by centrifugation with the Beckman SW28 rotor at 25,000 rpm (112,000 g) at 4° C. for 15 h. The pseudovirus identification involves performing pseudovirus infection in vitro by transfecting HEK-293FT cells overexpressing human angiotensin-converting enzyme 2 (hACE2) or transduced with empty lentivirus plasmid with the SARS-CoV-2 pseudovirus and a control pseudovirus encoding green fluorescent protein (GFP) into a 48-well plate for 48 h and 72 h, observing the pseudovirus infection under a fluorescence microscope and collecting a supernatant at 72 h, and detecting secretion of virus particles by a fluorescence quantitative PCR; determining efficiency of S protein fusion into the SARS-CoV-2 pseudovirus by western blotting with mouse monoclonal antibody anti-SARS-CoV-2 S (S2), and using a 2019-nCoV nucleic acid detection kit (Sansure Biotech Inc., China) to detect an ORFlab gene of the SARS-CoV-2 in the SARS-CoV-2 pseudovirus by RT-qPCR to ensure successful integration of a virus genome of the SARS-CoV-2 into a lentivirus. The carboxy magnetic beads and antibody coupling involves activating the carboxy magnetic beads continuously with an NHS solution and an EDC solution at 25° C. for 30 min to obtain activated carboxy magnetic beads MSP-COOH-F1; adding the activated carboxy magnetic beads MSP-COOH-F1 to a diluted antibody CQ25, mixing and rotating at 4° C. for 4 h, separating a supernatant to obtain an antibody-coupled magnetic bead complex, blocking the antibody-coupled magnetic bead complex with 1% BSA solution at 25° C. for 30 min; evaluating a coupling effect by the SDS-PAGE and Coomassie blue staining with the antibody-coupled magnetic bead complex and the separated supernatant. The affinity antibody screening involves performing SDS-PAGE and agarose gel electrophoresis respectively on a SARS-CoV-2 pseudovirus lysate and virus particles for resolution and membrane transfer, using mouse/human anti-SARS-CoV-2 S/M monoclonal antibodies as primary antibodies and horseradish peroxidase (HRP)-sheep anti-mouse monoclonal antibodies as secondary antibodies to screen an antibody with optimal specificity and affinity. The SARS-CoV-2 quantitative RT-qPCR detection involves mixing the antibody-coupled magnetic bead complex and the SARS-CoV-2 pseudovirus in a PBS buffer at room temperature for 45 min to obtain a captured complex, and detecting a SARS-CoV-2 RNA level of the captured complex by using a novel coronavirus nucleic acid detection kit in a Bio -Rad CFX96 system.

The disclosure provides a novel pseudovirus system that can completely simulate complete SARS-CoV-2, and provides a novel immune molecule detection method of complete SARS-CoV-2 virus particles on this basis. Through screening antibodies for capturing virus particles and optimizing antibody-magnetic bead coupling parameters and detection conditions, the immune molecule detection method of the disclosure can detect complete SARS-CoV-2 virus particles, and has high sensitivity and specificity. The anti-interference ability of the immune molecule detection of the disclosure to serum and HBV is more significant than that of direct qPCR detection, and the coefficients of variation of the analysis in the detection are 0.83% and 5.19% respectively, suggesting that the novel immune molecule detection method has good specificity and stability for detecting complete SARS-CoV-2 particles. The pseudo-SARS-CoV-2 virus of the disclosure contains three key points. Firstly, the pseudo-SARS-CoV-2 virus can express the active spike (S) glycoprotein of SARS-CoV-2 on the capsid of the pseudovirus, and trigger a specific immune reaction with the magnetic bead-antibody to capture complete virus particles. Secondly, three fragments of viral RNA sequence, the ORF1ab gene (15415-15540), the N gene (28750-29150) and the E gene (26360-26381), are encapsulated into the genome of the pseudovirus, which means that this new pseudovirus can be used in all nucleic acid detection reagents recommended and sold by world health organization (WHO) without redesigning the primers of qPCR. In addition, the GFP gene is integrated into the genome of the pseudovirus, which can conveniently evaluate the packaging efficiency of the pseudovirus and the ability of the pseudovirus to infect cells. Surprisingly, it is found that the new pseudovirus, like the normal pseudovirus, will not continue to proliferate and secrete into the supernatant to form a secondary infection after infecting cells, which means that the SARS-CoV-2 pseudovirus is very safe in subsequent experiments such as simulating virus infection and neutralizing antibody evaluation.

The method of the disclosure can be used in various situations, such as detecting that the clearance of complete virus particles, i.e., the disappearance of infectivity, evaluating whether the patients reinfected with virus can still infect the surrounding people after discharge, detecting whether there are complete virus particles in high-risk environments such as virus detection sampling rooms, international flight cabins, international cold chain logistics bases, and so on, so as to evaluate the infection risk and take corresponding protective measures. In the following research and application, the sensitivity of detection can be improved by screening antibodies with more affinity and carboxy magnetic beads with higher quality, and the isothermal nucleic acid amplification method can be combined with the method to further simplify the experimental process and reduce the dependence on experimental equipment, thus promoting the application of this detection method in clinical practice. In summary, the new pseudovirus fully simulates SARS-CoV-2, making the related research safer and more convenient. The method based on immune molecules is used to detect the complete virus particles for the first time, which can more accurately evaluate the infection status of patients and the risk of environmental infection, and realize personalized and accurate treatment and effective utilization of environmental resources.

### BRIEF DESCRIPTION OF DRAWINGS

FIG. 1 illustrates a flowchart for detecting virus particles by an immune-molecular method of the disclosure.

FIGS. 2A-2D illustrate detection results of hepatitis B virus (HBV) particles in supernatant of HepG 2.2.15 and HepAD38 cells by an immune molecule virus particle detection kit of the disclosure.

FIGS. 3A-3B illustrate detection results of HBV particles in serum of patients infected with HBV by an immune molecule virus particle detection kit of the disclosure.

FIGS. 4A-4C illustrate diagrams showing results of gradient optimization of antibodies required for coupling.

FIG. 5 illustrates a diagram of a verification result of virus capture.

FIGS. 6A-6B illustrate diagrams of verification results of a virus capture method.

FIG. 7 illustrates an optimization result diagram of a virus capture system of a BC group.

FIG. 8 illustrates an optimization result diagram of a virus capture system of a BS group.

FIGS. 9A-9B illustrate result diagrams of virus particle content in the HepG2.2.15 cells.

FIGS. 10A-10B illustrate result diagrams of virus particle content in the supernatant of the HepAD38 cells.

FIG. 11 illustrates a result diagram of virus particle content in serum samples.

FIGS. 12A-12E illustrate schematic diagrams of packaging and identification of severe acute respiratory syndrome coronavirus 2 (SARS-CoV-2) lentivirus. Specifically, FIG. 12A illustrates structures of SARS-CoV-2 and SARS-CoV-2 pseudovirus. FIG. 12B illustrates a packaging principle of the lentivirus. FIG. 12C illustrates infectivity of SARS-CoV-2 pseudovirus, where HEK-293FT cells transfected with empty plasmid and vesicular stomatitis virus G (VSV-G) pseudovirus encoding green fluorescent protein (GFP) are used as control. FIG. 12D illustrates a detection result of SARS-CoV-2 spike protein (S protein) in the lentivirus by western blotting, where SARS-CoV-2 S protein overexpressed in 293T cells transfected with a plasmid encoding wild-type SARS-CoV-2 S glycoprotein is used as control. FIG. 12E illustrates a detection result of complementary DNA (cDNA) of synthetic clone of SARS-CoV-2 lentivirus (Sansure Biotech Inc., Hunan, China) using a novel coronavirus (2019-nCoV) nucleic acid detection kit by quantitative polymerase chain reaction (qPCR), where the VSV-G pseudovirus prepared by the same procedure is use as a negative sample.

FIGS. 13A-13B illustrate diagrams identifying potential antibodies that bind to complete virus particles of SARS-CoV-2 pseudovirus. Specifically, in FIG. 13A, after the SARS-CoV-2 pseudovirus is heated at 100° C. for 10 minutes, the potential antibodies bound to the complete virus particles are identified by western blotting, where SARS-CoV-2 S protein expressed in 293T cells transfected with a plasmid encoding wild-type SARS-CoV-2 S glycoprotein is used as control. In FIG. 13B, the potential antibodies binding with the complete virus particles are identified through particle gel, where VSV pseudovirus prepared by the same method is used as a negative control.

FIGS. 14A-14F illustrate result diagrams of establishing a new SARS-CoV-2 pseudovirus detection platform based on immune capture. Specifically, FIG. 14A illustrates a flowchart of SARS-CoV-2 pseudovirus detection platform based on immune capture. FIG. 14B illustrates protein concentration of carboxy magnetic bead-antibody complexes after coupling determined by bicinchoninic acid (BCA) assay. FIG. 14C illustrates optimization of coupling parameters. FIG. 14D illustrates particle size analysis of carboxy magnetic beads (MB) and magnetic beads coupled with CQ25 antibodies (MB-CQ25), P<0.0001; ****. FIG. 14E illustrates captured SARS-CoV-2 pseudovirus identified by anti-hiv1 P24 antibodies. FIG. 14F illustrates specificity identification of virus captured by CQ25 antibody-carboxy magnetic bead complex.

FIGS. 15A-15D illustrate verification result diagrams of SARS-CoV-2 pseudovirus detection platform. Specifically, FIG. 15A illustrates linear regression analysis, when the titer of SARS-CoV-2 pseudovirus is in a range of 10² to 10⁷ transducing units per milliliter (TU/mL), the quantification Cq value detected by immune molecules has a linear relationship with the titer (log transformation), y=−2.57x+40.203, R²=0.99; and results directly detected qPCR are in the range of 10 to 10⁷ TU/mL, y=−2.070x+33.23, R²=0.98. FIG. 15B illustrates specificity of detection, 24 carboxy magnetic beads-CQ25 antibody complexes (CQ25-MB) and 24 captured pseudoviruses are used to determine the specificity of this experiment, P<0.0001, ****. FIG. 15C illustrates VSV-G pseudovirus interference; Adding VSV-G pseudoviruses with different volumes to the detection platform to detect interference. FIG. 15D illustrates interference of different copies of HBV in serum, where the titer of SARS-CoV-2 pseudovirus is 10⁵ TU/mL.

### DETAILED DESCRIPTION OF EMBODIMENTS

The disclosure will be described in detail through specific embodiments, and it is pointed out that the following embodiments are only used for further explanation of the disclosure, and cannot be understood as limiting the scope of protection of the disclosure. Those skilled in the art can make some non-essential modifications and adjustments to the disclosure based on the above summary. The raw materials and reagents used in the disclosure are all commercially available products. Unless otherwise specified, the percentages used in the disclosure are all weight percentages.

Cell lines: HepG2.2.15 and HepAD38 cell lines are from the Key Laboratory of Molecular Biology of Infectious Diseases of Chongqing Medical University, which are long-term stored.

Serum samples are from Yubei District People's Hospital of Chongqing.

Main reagents: carboxy magnetic bead suspension (from Chongqing Farsighted-Blue-Dragon Biotechnology Co., Ltd., China); HBV nucleic acid assay kit, HBV ribonucleic acid (HBV RNA) quantitative detection kit (PCR-fluorescence probe method) (from Sansure Biotech Inc., Hunan, China); biotin, dimethyl sulfoxide (DMSO), N-Hydroxysuccinimide ester (NHSB), 4-morpholinoethanesulfonic acid (MES), deoxyribonuclease, bovine serum albumin (BSA) (from Sangon Biotech (Shanghai) Co., Ltd.); DNA maker (from Tsingke Biotech Co., Ltd., China); Goldview nucleic acid dye (from Thermo Fisher Scientific Inc.); PreS1 monoclonal antibody, HBc monoclonal antibody (from Xiamen Innodx Biotechnology Co., Ltd.); phosphate buffered saline (PBS) powder (from Beijing Leagene Biotechnology Co., Ltd.), streptavidin, N-hydroxysuccinimide (NHS), and 1-Ethyl-3-[3-dimethylaminopropyl]carbodiimide hydrochloride (EDC) (from Chongqing Farsighted-Blue-Dragon Biotechnology Co., Ltd.)

### Preparation of Main Reagents

| Name | Preparation method |
|---|---|
| NHS, EDC | Freshly prepared, each weighing 0.0125 grams (g), and |
| | dissolved in 500 microliters (μL) MES buffer. |
| Blocking | 0.05 g BSA, dissolved in 1 milliliter (mL) MES buffer. |
| solution | |
| PBS buffer | 20 millimoles per liter (mM) PBS, pH 7.4, stored at |
| | 4° C., used at room temperature. |
| Preservation | 20 mM PBS (pH 7.0) + 0.09% sodium azide + 0.01% |
| solution | polysorbate 20, stored at 4° C. |
| Antibody solution | Antibodies are diluted with MES buffer to a final |
| used for coupling | concentration of 0.6 grams per liter (g/L). |
| Decolorizing | 100 mL acetic acid + 200 mL ethanol + 700 mL |
| solution | double-distilled water (ddH₂O). |
| Coomassie | 100 milligrams (mg) Coomassie brilliant blue G-250 + |
| brilliant blue | 50 mL 90% ethanol + 100 mL 85% phosphoric acid, |
| | fix volume to 1 L. |

### 1. Immune Molecule Virus Particle Detection Kit

### Embodiment 1 Biotin Modification of Monoclonal Antibodies

The monoclonal antibodies to be biotinylated are diluted to 1 mg/mL with 0.1 moles per liter (mol/L) sodium bicarbonate buffer (pH 8.0) or 0.5 mol/L boric acid buffer (pH 8.6), and the biotinylated volume for general laboratory application is 1-2.5 mL. Then, the monoclonal antibodies are fully dialyzed with 0.1 mol/L sodium bicarbonate buffer (pH 8.0) or 0.5 mol/L boric acid buffer (pH 8.6). 1 mg biotin is dissolved with 1 mL DMSO to obtain a biotin solution. 120 microliters (μL) biotin solution (containing 120 micrograms abbreviated as μg biotin) are added to 1 mL monoclonal antibody solution (containing 1 mg monoclonal antibodies), and continuously stirred at room temperature and kept the temperature for 2-4 hours. After that, 9.6 μL 1 mol/L ammonium chloride (NH₄Cl) is added (1 μL NH₄Cl for every 25 μg NHSB) and stirred at room temperature for 10 minutes. At 4° C., PBS is fully dialyzed to remove free biotin; the sample is loaded on a 1 mL molecular sieve column, slowly eluted with PBS, collected 1 mL/tube, and the proteins are washed down between 1-3 mL. Finally, sodium azide (final concentration 0.5 g/L) and 1.0 g/L BSA are added to the sample to obtain a binding product. The binding product is stored at 4° C. in the dark, or 50% redistilled glycerol is added and stored at −20° C.

### Embodiment 2 Magnetic Beads Coupled with Streptavidin

The carboxy magnetic beads are vigorously shaken to make them disperse evenly. 3.3 mg magnetic beads are put into a 2 mL EP tube, separated by magnetic force, and washed with precooled MES buffer for 3 times. After that, a magnetic field is applied, the supernatant is removed, 100 μL NHS and the same amount of EDC solution are quickly added into the EP tube, shaken vigorously, and the magnetic beads are continuously activated for 30 minutes at 25° C. With the help of a magnetic rack, the magnetic beads are washed with the precooled MES buffer for 3 times and used for streptavidin coupling as soon as possible. The streptavidin to be coupled is diluted with the precooled MES buffer until the final antibody concentration is about 0.6 g/L, 100 μL. The activated magnetic beads are resuspended with 100 μMES buffer and shaken vigorously to ensure that the magnetic beads are completely dispersed. 20 μL of activated magnetic bead suspension is taken each time, the activated magnetic bead suspension is slowly added into the diluted streptavidin suspension for 5 times and immediately gently mixed evenly after adding magnetic beads, and gently rotated and mixed evenly at 4° C. for 4 hours. 5% BSA solution (10 mL MES buffer+0.5 g BSA) is prepared. A magnetic field is applied, the supernatant of the mixed solution is removed, 200 μL BSA blocking solution is quickly added into the tube, and gently rotated at 25° C. for 30 minutes. With the help of a magnetic rack, the magnetic beads are washed with PBS for three times, 120 μL of preservation solution is transferred into the tube, the magnetic beads coupled with streptavidin are suspended and stored at 4° C.

### Embodiment 3 Monoclonal Antibodies of Viral Envelope Protein Coupled with Magnetic Beads

The carboxy magnetic beads are vigorously shaken to make them disperse evenly. 3.3 mg magnetic beads are put into a 2 mL EP tube, separated by magnetic force, and washed with precooled MES buffer for 3 times. After that, a magnetic field is applied, the supernatant is removed, 100 μL NHS and the same amount of EDC solution are quickly added into the EP tube, shaken vigorously, and the magnetic beads are continuously activated for 30 minutes at 25° C. With the help of a magnetic rack, the magnetic beads are washed with the precooled MES buffer for 3 times and used for monoclonal antibodies coupling as soon as possible. The monoclonal antibodies to be coupled is diluted with the precooled MES buffer until the final antibody concentration is about 0.6 g/L, 100 μL. The activated magnetic beads are resuspended with 100 μL MES buffer and shaken vigorously to ensure that the magnetic beads are completely dispersed. 20 μL of activated magnetic bead suspension is taken each time, the activated magnetic bead suspension is slowly added into the diluted monoclonal antibodies suspension for 5 times and immediately gently mixed evenly after adding magnetic beads, and gently rotated and mixed evenly at 4° C. for 4 hours. 5% BSA solution (10 mL MES buffer+0.5 g BSA) is prepared. A magnetic field is applied, the supernatant of the mixed solution is removed, 200 μL BSA blocking solution is quickly added into the tube, and gently rotated at 25° C. for 30 minutes. With the help of a magnetic rack, the magnetic beads are washed with PBS for three times, 120 μL of preservation solution is transferred into the tube, the magnetic beads coupled with monoclonal antibodies are suspended and stored at 4° C.

### Embodiment 4 Antibody-Coupled Magnetic Beads Capture of Hepatitis B Virus (HBV) Particles in Cell Supernatant

5 μL magnetic bead preservation solution coupled HBV envelope PreS1 antibodies (or HBV core particle HBc antibodies) is taken and magnetically separated, the supernatant is discarded, and washed twice with PBS. 5 μL cell supernatant (HepG2.2.15 or HepAD38 cells) is taken and added to a 2 mL EP tube, diluted with PBS until the system is 500 μL, added antibody-magnetic bead conjugates, mixed evenly, and rotate and bound at 25° C. for 40 minutes. After the magnetic field is applied, the supernatant is discarded. The washed capture is resuspended with PBS, and then analyzed by western blotting and qPCR according to different purposes.

### Embodiment 5 Binding of HBV Particles in Cell Supernatant with Biotin-Labeled Antibody and Captured by Streptavidin-Coupled Magnetic Beads

5 μL cell supernatant (HepG2.2.15 or HepAD38 cells) is taken to a 2 mL EP tube, and diluted with PBS until the system is 500 μL. The coupled biotin-modified monoclonal antibodies (HBV envelope PreS1 antibodies or HBV core particle HBc antibodies) are added, incubated and bound, and rotated and bound at 25° C. for 10 minutes. 5 μL coupled streptavidin magnetic bead preservation solution is taken and magnetically separated, the supernatant is discarded, and washed twice with PBS. The streptavidin-coupled magnetic beads are added to the prepared sample, mixed evenly, and rotated and bound at 25° C. for 40 minutes. After the magnetic field is applied, the supernatant is discarded. The washed capture is resuspended with PBS, and then analyzed by western blotting and qPCR according to different purposes.

### Embodiment 6 Antibody-Coupled Magnetic Bead Capture of Novel Coronavirus Particles or Novel Coronavirus Pseudovirus

5 μL antibody-coupled magnetic bead preservation solution (novel coronavirus spike antigen or receptor-binding domain abbreviated as RBD antibodies) is taken and magnetically separated, the supernatant is discarded, and washed twice with PBS. 5 μL novel coronavirus particle (or novel coronavirus pseudovirus) solution is taken and added to a 2 mL EP tube, and diluted with PBS until the system is 500 μL. The antibody-coupled magnetic beads are added to the prepared sample, mixed evenly, and rotated and bound at 25° C. for 40 minutes. After the magnetic field is applied, the supernatant is discarded. The washed capture is resuspended with PBS, and then analyzed by western blotting and qPCR according to different purposes.

### Embodiment 7 Binding of Novel Coronavirus Particles (or Novel Coronavirus Pseudovirus) with Biotin-Labeled Antibodies and captured by Streptavidin-Coupled Magnetic Beads

5 μL novel coronavirus particles (or novel coronavirus pseudovirus) are taken and added to a 2 mL EP tube, and diluted with PBS until the system is 500 μL. The coupled biotin-modified monoclonal antibodies (novel coronavirus spike antigen or RBD antibodies) are added, incubated and bound, and rotated and bound at 25° C. for 10 minutes. 5 μL streptavidin-coupled magnetic bead preservation solution is taken and magnetically separated, the supernatant is discarded, and washed twice with PBS. The streptavidin-coupled magnetic beads are added to the prepared sample, mixed evenly, and rotated and bound at 25° C. for 40 minutes. After the magnetic field is applied, the supernatant is discarded. The washed capture is resuspended with PBS, and then analyzed by western blotting and qPCR according to different purposes.

### Embodiment 8 Antibody-Coupled Magnetic Beads Capture of HBV Particles in Supernatant of Patients Infected with HBV

(1) 5 μL antibody-coupled magnetic bead preservation solution (HBV envelope PreS1 antibodies or HBV core particle HBc antibodies) is taken, magnetically separated to discard the supernatant, and washed twice with PBS.
(2) 5 μL serum is sampled from patients infected with HBV and added to a 2 mL EP tube, and diluted with PBS until the system is 500 μL, so as to obtain a prepared sample.
(3) The antibody-coupled magnetic beads are added to the sample prepared in the step (2), mixed evenly, and rotated and bound at 25° C. for 40 minutes.
(4) after applying a magnetic field, the supernatant discarded. The washed capture is resuspended with PBS, and then analyzed by western blotting and qPCR according to different purposes.

### Embodiment 9 Binding of HBV Particles in Supernatant of Patients Infected with HBV with Biotin-Labeled Antibodies and Captured by Streptavidin-Coupled Magnetic Beads

5 μL serum is sampled from patients infected with HBV and added to a 2 mL EP tube, and diluted with PBS until the system is 500 μL. The coupled biotin-modified monoclonal antibodies (HBV envelope PreS1 antibodies or HBV core particle HBc antibodies) are added, incubated and bound, and rotated and bound at 25° C. for 10 minutes. 5 μL streptavidin-coupled magnetic bead preservation solution is taken and magnetically separated, the supernatant is discarded, and washed twice with PBS. The streptavidin-coupled magnetic beads are added to the prepared sample, mixed evenly, and rotated and bound at 25° C. for 40 minutes. After the magnetic field is applied, the supernatant is discarded. The washed capture is resuspended with PBS, and then analyzed by western blotting and qPCR according to different purposes.

### Embodiment 10 Antibody-Coupled Magnetic Beads Capture of Hepatitis C Virus (HCV) Particles in Supernatant of Patients Infected with HCV

5 μL antibody-coupled magnetic bead preservation solution (HCV envelope protein E2, E3 antibodies or HCV core antibodies) is taken and magnetically separated, the supernatant is discarded, and washed twice with PBS. 5-500 μL serum is sampled from patients infected with HCV and added to a 2 mL EP tube, and diluted with PBS until the system is 500 μL. The antibody-coupled magnetic beads are added to the prepared sample, mixed evenly, and rotated and bound at 25° C. for 40 minutes. After the magnetic field is applied, the supernatant is discarded. The washed capture is resuspended with PBS, and then analyzed by western blotting and qPCR according to different purposes.

### Embodiment 11 Binding of HCV Particles in Supernatant of Patients Infected with HCV with Biotin-Labeled Antibodies and Capture of Streptavidin-Coupled Magnetic Beads

5-500 μL serum is sampled from patients infected with HCV and added to a 2 mL EP tube, and diluted with PBS until the system is 500 μL.

The coupled biotin-modified monoclonal antibodies (HCV envelope protein E2, E3 antibodies or HCV core antibodies), incubated and bound, and rotated and bound at 25° C. for 10 minutes. 5 μL streptavidin-coupled magnetic bead preservation solution is taken and magnetically separated, the supernatant is discarded, and washed twice with PBS. The streptavidin-coupled magnetic beads are added to the prepared sample, mixed evenly, and rotated and bound at 25° C. for 40 minutes. After the magnetic field is applied, the supernatant is discarded. The washed capture is resuspended with PBS, and then analyzed by western blotting and qPCR according to different purposes.

### Embodiment 12 Antibody-Coupled Magnetic Beads Capture of Human Immunodeficiency Virus (HIV) (or Pseudovirus) Particles

(1) 5 μL coupled antibodies (HIV envelope protein gp120 and gp41 antibodies or virus core formed by HIV p17 and P24) are taken and magnetically separated to discard the supernatant, and washed twice with PBS.
(2) 5-500 μL of serum from patients infected with HIV or supernatant of HIV-secreting cells is taken and added into a 2 mL EP tube, and diluted with PBS until the system is 500 μL.
(3) The antibody-coupled magnetic beads are added to the sample prepared in the step (2), mixed evenly, and rotated and bound at 25° C. for 40 minutes.
(4) after applying the magnetic field, the supernatant is discarded. The washed capture is resuspended with PBS, and then analyzed by western blotting and qPCR according to different purposes.

### Embodiment 13 Binding of HIV (or Pseudovirus) Particles with Biotin-Labeled Antibodies and Capture of Streptavidin-Coupled Magnetic Beads

5-500 82 L of serum from HIV infected patients or supernatant of HIV-secreting cells is taken and added into a 2 mL EP tube, and diluted with PBS until the system is 500 μL. The coupled biotin-modified monoclonal antibodies (HIV envelope protein gp120 and gp41 antibodies or virus core formed by HIV p17 and P24), incubated and bound, and rotated and bound at 25° C. for 10 minutes. 5 μL streptavidin-coupled magnetic bead preservation solution is taken and magnetically separated to discard the supernatant, and washed twice with PBS. The streptavidin-coupled magnetic beads are added to the prepared sample, mixed evenly, and rotated and bound at 25° C. for 40 minutes. After the magnetic field is applied, the supernatant is discarded. The washed capture is resuspended with PBS, and then analyzed by western blotting and qPCR according to different purposes.

### Embodiment 14 Detection of DNA Virus Particles by Real-Time Fluorescence Quantitative PCR

The virus particles are captured according to the above method, separated by magnetic force and washed twice with 200 μL PBS. The captured complex is resuspended with 50 μL PBS, transferred to 8-strip PCR tubes, and the supernatant is removed with the help of a magnetic rack of PCR plate. The required reagents are placed at room temperature in advance to avoid light, which is convenient for subsequent use. PCR mixed solutions are prepared, per person, 38 μL reaction solution+2 μL enzyme mixed solution+0.2 μL internal standard; and qPCR parameters are set.

| | | | | Cycle |
|---|---|---|---|---|
| Procedures | Temperature | Time | | number |
| Uracil N-Glycosylase (UNG) reaction | 50° C. | 2 | min | 1 |
| Taq enzyme activation | 94° C. | 5 | min | 1 |
| Denaturation | 94° C. | 15 | s | 45 |
| Annealing, extension, | 57° C. | 30 | s | 45 |
| fluorescence detection | | | | |
| Instrument cooling | 25° C. | 10 | s | 1 |

### Embodiment 15 Detection of RNA Virus Particles by Real-Time Fluorescence Quantitative PCR

(1) The virus is captured by the above method, separated by magnetic force, and washed twice with 200 μL PBS.
(2) Then, nucleic acid extraction is performed using a nucleic acid extraction kit.
(3) DNA digestion and DNA enzyme inactivation: DNA digestion is carried out by using ribonucleic acid.
(4) Standards A-D, negative and positive controls are set, 5 μL sample release agent is added to each hole of the 8-strip tubes, instantly centrifuged, beaten and mixed evenly, and allowed to stand in the dark for 10 minutes.
(5) PCR mixed solutions are prepared, per person, 38 μL reaction solution+2 μL enzyme mixed solution+0.2 μL internal standard.
(6) qPCR cycle parameters are set.

| | | Temper- | | | Cycle |
|---|---|---|---|---|---|
| | Procedures | ature | Time | | number |
| 1 | Pre-denaturation and | 95° C. | 1 | min | 1 |
| | enzyme activation | | | | |
| 2 | Reverse transcription | 60° C. | 30 | min | 1 |
| 3 | cDNA pre-denaturation | 94° C. | 1 | min | 1 |
| 4 | Denaturation | 95° C. | 15 | s | 45 |
| | Annealing, extension and | 60° C. | 30 | s | |
| | fluorescence acquisition | | | | |
| 5 | Instrument cooling | 25° C. | 10 | s | 1 |

### Results

In order to specifically separate different virus particles from virus-infected people or corresponding cell supernatant, the disclosure developed an immune method based on antigen-antibody interaction to capture and separate different virus particles. PreS1 is considered to be a unique structure of HBV Dane, and the genome of NC particles is directly encapsulated by HBc protein to form a nucleocapsid without being enveloped, so it can be recognized by HBc monoclonal antibodies. Hepatitis A virus (HAV), hepatitis B virus (HBV), hepatitis C virus (HCV), hepatitis D virus (HDV), hepatitis E virus (HEV), novel coronavirus (SARS-CoV-2), human immunodeficiency virus (HIV), influenza virus, partial pulmonary virus, human papillomavirus (HPV), herpes virus, herpesvirus hominis, Zika virus, Ebola virus (EBV), human T-lymphocytic virus, avian influenza virus, hog cholera virus (CSFV), poliovirus, rabies virus, adenovirus, lentivirus, and other common viruses, whose complete virus particles and core particles also have similar characteristics.

In view of this, the inventor uses “virus particle-antibody-biotin-streptavidin-magnetic bead capture” and “virus particle-antibody-magnetic bead capture” respectively to realize that complete virus particles and empty-shell viruses, free envelope antigens can be separated from other virus components (protein-virus RNA/DNA complexes, free virus gene fragments, etc.) and other sub-viral particle components.

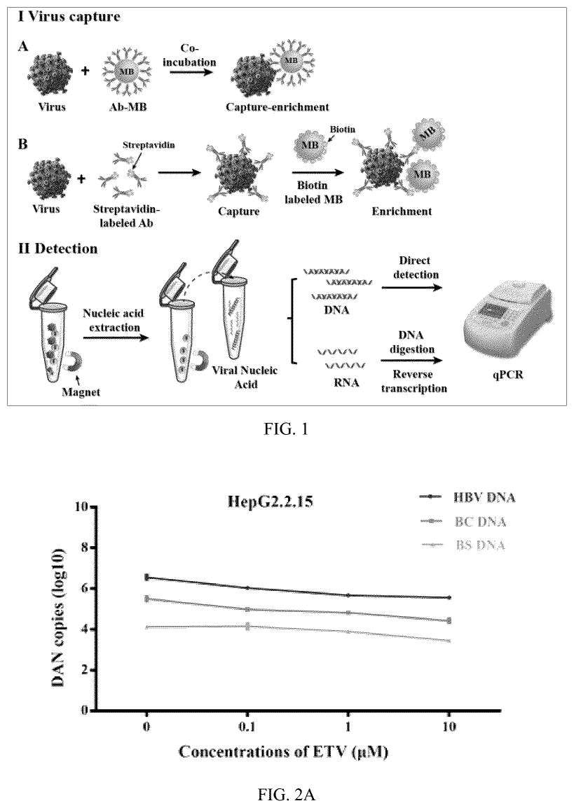

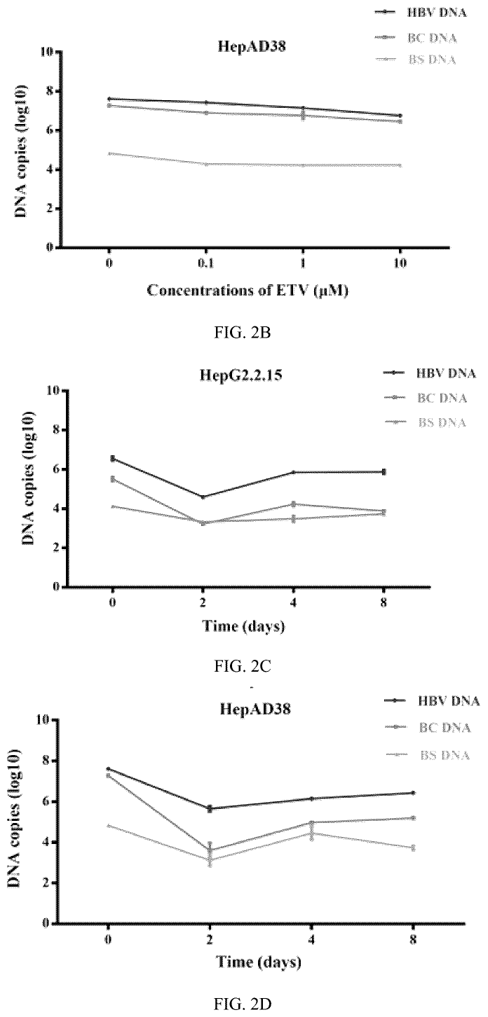

The inventor selects the supernatant of HBV-secreting cells (HepG2 and HepAD38) for testing and verification. HepG2.2.15 and HepAD38 are the two cell lines that are commonly used in the laboratory to stably express HBV, and entecavir (ETV) is the first-line drug in clinical treatment of hepatitis B at present. The inventor uses different concentration gradients of ETV (0.1 μM, 1 μM, 10 μM, PBS as control group). Then, the virus particles are captured by virus particle-antibody (the envelope PreS1 monoclonal antibody and the core particle HBc monoclonal antibody)-biotin-streptavidin-magnetic beads, and the genomic DNA of HBV in the corresponding virus particles is detected. The results show that the viral genomic DNA of complete virus particles and core particles could be detected in the cell supernatant, which is negatively correlated with the total viral genomic DNA in the cell supernatant and the treatment concentration of ETV. The higher the concentration of ETV, the lower the viral genomic DNA detected by the three methods (FIGS. 2A-2B). In addition, the inventor treats HepG2 and HepAD38 cells with ETV (1 uM) individually, and finds that the total HBV genomic DNA, complete virus particle genomic DNA and virus core particle DNA all decreased rapidly in two days, and the levels of the three virus genomic DNA all rose with the degradation of drug concentration and other factors in the later period (FIGS. 2C-2D).

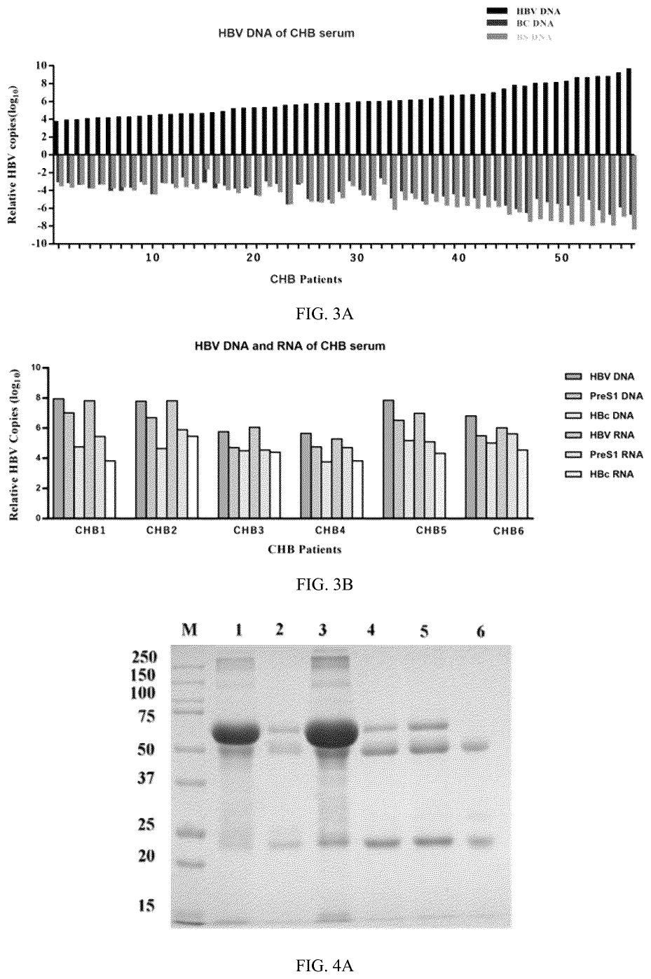

HBV is a DNA virus, and its virus core particles contain viral genomic DNA. Studies have found that pregenomic RNA (pgRNA) without reverse transcription in the nucleocapsid of HBV. In view of the fact that the inventor's technical solution can be used to detect DNA in virus particles, it can also detect viral RNA in virus particles. The inventor selects the serum of clinical patients infected with HBV to detect viral DNA and viral RNA in HBV particles. The inventor uses “virus particle-antibody-biotin-streptavidin-magnetic bead capture” and “virus particle-antibody-magnetic bead capture” respectively to capture complete virus particles and core particles respectively, and then quantitative PCR and reverse transcription-quantitative PCR are used to detect virus DNA and RNA respectively. The results show that (FIG. 3A), except for individual patients, in the serum of 56 patients with HBV infection, compared with the HBV core particle virus DNA group (BC DNA), the content of complete virus particle DNA (BS DNA) in serum is higher, especially at high titer serum load. This phenomenon is particularly obvious, and the content of complete virus particles in serum increases significantly with the increase of HBV DNA copies in serum. The sera of six patients infected with HBV are detected, the viral RNA exists in both complete and nucleocapsid particles, the RNA content of the complete virus particles is much higher than that of the nucleocapsid particles, which is similar to the DNA in virus particles (FIGS. 3A and 3B).

### 2. Detection of immune capture molecules of complete HBV particles

### Embodiment 16

### Antibody-Magnetic Bead Coupling

(1) Carboxy magnetic beads are shaken vigorously to make them disperse evenly. 3.3 mg magnetic beads are put into a 2 mL EP tube, separated by a magnetic force, and washed with a precooled MES buffer for 3 times.
(2) A magnetic field is applied to remove the supernatant, 100 μL NHS and the same amount of EDC solution are quickly added in the EP tube, shaken vigorously, and continuously activated the magnetic beads at 25° C. for 30 minutes.
(3) With the help of a magnetic rack, the magnetic beads are washed with the precooled MES buffer for three times and used for antibody coupling as soon as possible.
(4) Antibodies to be coupled are diluted with the precooled MES buffer until a final antibody concentration is about 0.6 g/L, 100 μL.
(5) The activated magnetic beads are resuspended with 100 μL MES buffer and shaken vigorously to ensure that the magnetic beads are completely dispersed.
(6) 20 μL activated magnetic bead suspension is taken each time, the activated magnetic bead suspension is slowly added into the diluted antibody suspension for 5 times, and gently mixed immediately after adding the activated magnetic beads, and gently rotated and mixed at 4° C. for 4 hours.
(7) 5% BSA blocking solution (10 mL MES buffer+0.5 g BSA) is prepared.
(8) The magnetic field is applied to remove the supernatant, 200 μL BSA blocking solution is quickly added into the EP tube, and gently rotated at 25° C. for 30 minutes.
(9) With the help of a magnetic rack, the magnetic beads are washed with PBS for three times.
(10) 120 μL preservation solution is transferred into the EP tube, suspended the magnetic beads and stored at 4° C.

### Capture of HBV Particles

(1) 5 μL antibody-coupled magnetic bead preservation solution is taken and magnetically separated to discard the supernatant, and washed twice with PBS.
(2) 5 μL cell supernatant or serum of patients with HBV is taken and added to a 2 mL EP tube, and diluted with PBS until the system is 500 μL.
(3) The antibody-coupled magnetic beads are added to the sample prepared in the step (2), mixed evenly, and rotated and bound at 25° C. for 40 minutes.
(4) After the magnetic field is applied to discard the supernatant, the washed capture is resuspended with PBS, and then analyzed by western blotting and qPCR according to different purposes.

### Real-Time Fluorescence Quantitative PCR

(1) The virus is captured by the above method, separated by a magnetic force, and washed twice with 200 μL PBS.
(2) The captured complex is resuspended with 50 μL PBS, then transferred to 8-strip PCR tubes, and the supernatant is removed with the help of a magnetic rack of PCR plate;
(3) The required reagents are placed at room temperature in advance to avoid light, which is convenient for subsequent use.
(4) Standards A-D, negative and positive controls are set, 5 μL of sample release agent is added to each hole of the 8-strip tubes, instantly centrifuged, beaten and mixed evenly, and allowed to stand in the dark for 10 minutes.
(5) PCR mixed solutions are prepared, per person, 38 μL reaction solution+2 μL enzyme mixed solution+0.2 μL internal standard.
(6) qPCR cycle parameters are set.

| | | | | Cycle |
|---|---|---|---|---|
| Procedures | Temperature | Time | | number |
| Uracil N-Glycosylase (UNG) reaction | 50° C. | 2 | min | 1 |
| Taq enzyme activation | 94° C. | 5 | min | 1 |
| Denaturation | 94° C. | 15 | s | 45 |
| Annealing, extension, | 57° C. | 30 | s | 45 |
| fluorescence detection | | | | |
| Instrument cooling | 25° C. | 10 | s | 1 |

(7) Running.

### Nucleic Acid Electrophoresis

1% nucleic acid gel is prepared; the qPCR products are taken as samples and all of them are loaded. Constant pressure 120 voltages (V) for 20 minutes, Analysis of ultraviolet photography of gel imager is performed.

### Results

### Virus Capture Principle

PreS1 is considered to be a unique structure of HBV Dane, and the genome of NC particles is directly encapsulated by HBc protein to form a nucleocapsid without being enveloped, so it can be recognized by HBc monoclonal antibodies. Based on this principle, the inventor incubates the serum of HBV patients with carboxy magnetic beads coupled with PreS1 and HBc mouse monoclonal antibodies respectively. In a proper proportion, the monoclonal antibodies and the corresponding antigens on the envelope protein or capsid protein form complexes, which are separated by the magnetic force, and the supernatant is discarded. In this situation, the virus is successfully captured on the magnetic beads, and the corresponding buffer resuspends the magnetic beads for subsequent related experimental research.

### Optimization of Antibody Concentration Coupled with Magnetic Beads

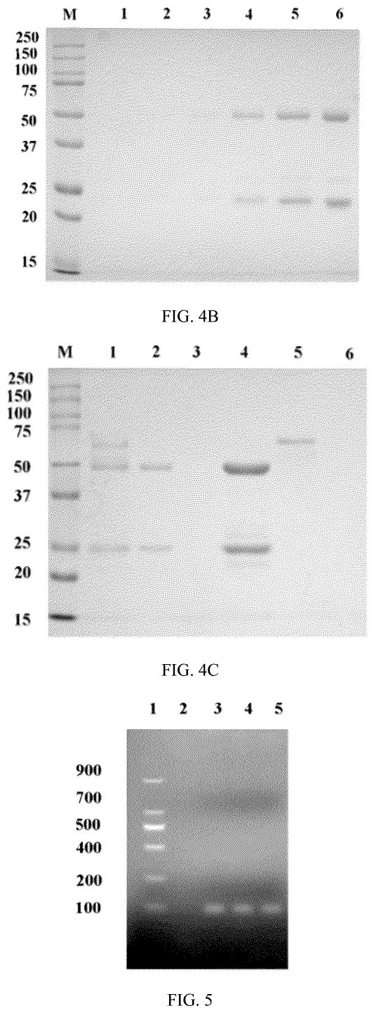

Different antibodies have different concentrations when they are saturated with magnetic beads because of their different types and amino groups. In order to avoid the difference of antigen capture caused by the different amount of antibody coupled with magnetic beads, the required concentration of antibody coupled with carboxy magnetic beads is optimized at first. The concentrations of magnetic beads, PreS1 antibody and HBc antibody used in this experiment are 2 mg/mL, 1 mg/mL and 2.7 mg/mL respectively. According to the recommendation of instructions of commercial carboxy magnetic beads, 18 μg antibodies can saturate 1 mg carboxy magnetic beads. Based on this standard, 18 μg, 36 μg, 54 μg, 72 μg, 90 μg and 108 μg of HBc antibodies are coupled with 1 mg of carboxy magnetic beads individually, and the HBc antibody-magnetic bead conjugate and the supernatant after antibody coupling are sampled, and the results are analyzed after Coomassie brilliant blue staining. As shown in FIG. 4A, when the amount of antibody added increased, the HBc antibody band detected in the complex gradually deepened. In FIG. 4B, when the amount of HBc antibody is 54 μg (lane 3), antibody band begins to appear in the supernatant, and the depth trend of bands is consistent with the total amount of added antibodies and the amount of Ab in the complex, suggesting that 54 μg HBc antibody can saturate 1 mg carboxy magnetic beads. Based on the principle of saving reagents, 54 μg HBc antibody is selected to couple with carboxy magnetic beads. Based on the same optimization method, FIG. 4C shows that the amount of PreS1 antibody required to saturate 1 mg carboxy magnetic beads is approximately 18 μg.

### Verification of HBV Virus Capture Method

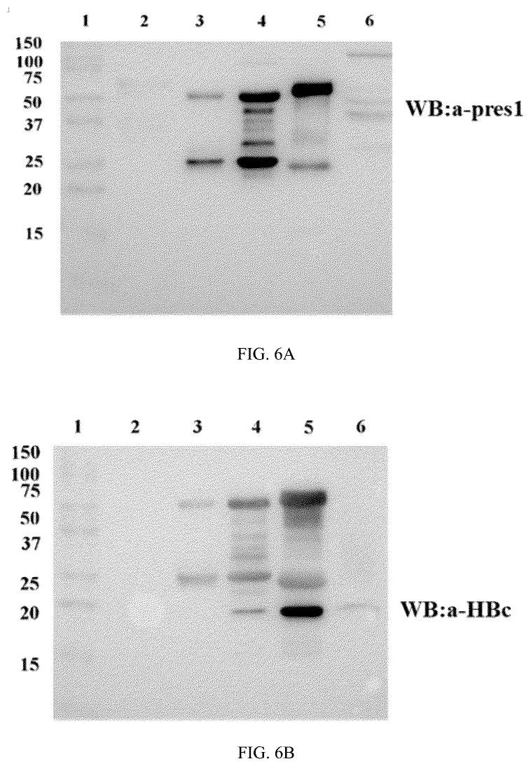

In order to verify whether the magnetic beads (coupled with antibodies) can effectively capture nucleic acid virus particles in HBV serum, the captured products are quantified by qPCR, and then nucleic acid electrophoresis is carried out with qPCR products as samples. As shown in FIG. 5, the nucleic acid results show that there is no objective band in BP group (lane 2: virus captured by magnetic beads coupled with unrelated antibodies), while the objective bands appeared at about 100 base pairs (bp) in BS group (lane 3: the virus captured by magnetic beads coupled with PreS1 antibodies), BC group (lane 4: the virus captured by magnetic beads coupled with HBc antibodies) and positive control group (lane 5). In summary, the capture system established in this project is feasible. In order to further verify the specificity of this method, the specific antibodies of recombinant GST-PreS1 protein and HBc protein are used as probes to detect HBV virus components in the captured by western blotting. NCs particles of HBV DNA type are mainly composed of HBc protein and HBV DNA. In contrast, HBV Dane particles also contain the outermost surface proteins (spike protein abbreviated as S protein, membrane glycoprotein abbreviated as M protein, polymerase abbreviated as L protein). As shown in FIGS. 6A-6B, compared with the negative control BP group, the BS group detected PreS1 protein at 42 kilodalton (kDa) (lane 4 in FIG. 6A) (because GST-PreS1 is unstable and easy to degrade, so the antibody purity is slightly lower), and HBc protein is detected at about 20 kDa (lane 4 in FIG. 6B), indicating that the virus particles captured by the BS group contain HBV large surface (LHBs) proteins and HBc proteins. Combined with the results of nucleic acid electrophoresis, it is suggested that the virus particles captured by the BS group are HBV Dane particles. However, the objective band is only detected at about 20 kDa in BC group (lane 5 FIG. 6B), suggesting that BC group can capture NCs particles. Generally speaking, the new capture system developed in this disclosure is not only feasible, but also has high specificity.

### Optimization of HBV Virus Capture System

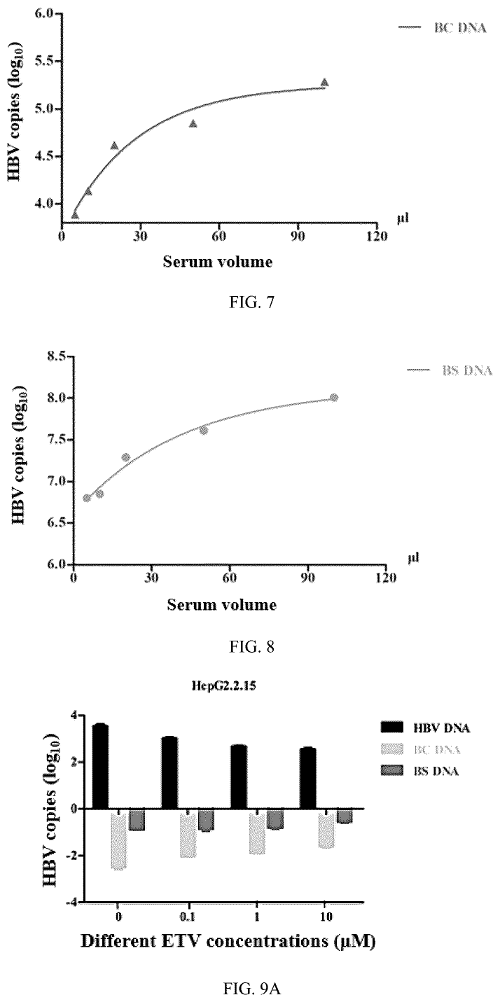

Based on the proportionality principle of antigen-antibody reaction, before analyzing the content of HBV virus particles in serum, the optimal proportion of antigen-antibody reaction should be optimized to ensure that virus particles with different components in serum samples are completely captured. A series of gradient volumes (5 μL, 10 μL, 20 μL, 50 μL, 100 μL) of 10⁷ HBV DNA patients' sera are taken as samples, 5 μL of antibody-magnetic bead complex is added, the virus is captured for 40 minutes, and a magnetic field is applied. After the magnetic beads are washed with PBS, qPCR analysis is performed on the captured samples with the help of an HBV nucleic acid quantitative kit (Sansure Biotech Inc.). As can be seen from FIG. 7 and FIG. 8, the HBV copies of the captured products in BC group and BS group increased with the increase of the added serum volume, indicating that 5 μL antibody-magnetic beads can completely capture the corresponding virus particles in the serum. Based on the principle of trace amount, a uniform capture system will be used in subsequent experiments: 5 μL HBV sample (diluted with 500 μL PBS) and 5 μL antibody-magnetic bead suspension.

### Content of Different HBV Virus Particles in Cell Supernatant

In order to study the dynamic changes of HBV DNA with different components, two cell lines, HepG2.2.15 and HepAD38, which stably express HBV, are pretreated with different concentration gradients of ETV (0.1 μM, 1 μM, 10 μM and a PBS control group), and the supernatant is discarded after 24 hours, and replaced with fresh Dulbecco's modified eagle medium (DMEM) and ETV with different concentration gradients. After 72 hours, the supernatant is transferred to an EP tube, and the virus particles in the supernatant are captured by PreS1 antibody-magnetic beads and HBc antibody-magnetic beads respectively, and the captured products are detected by qPCR. The detection results show that the copies of HBV

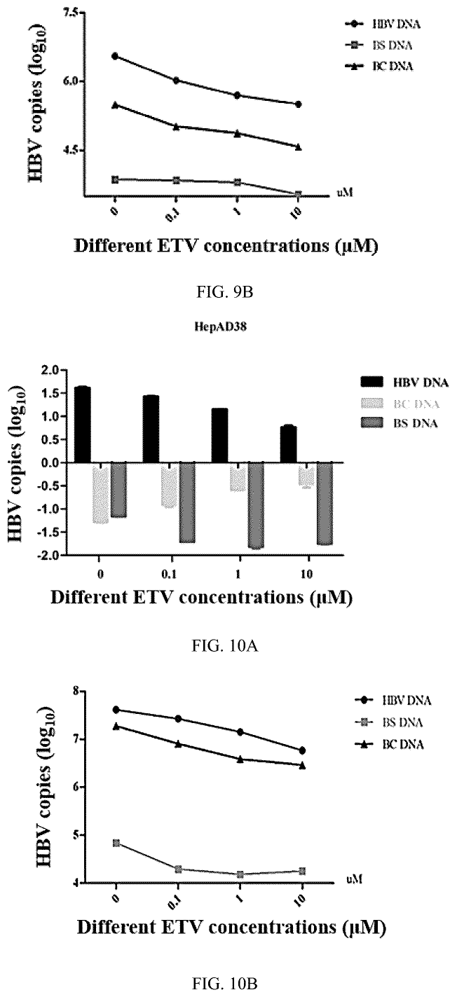

DNA in the supernatant decreased in a dose-dependent manner when HepG2.2.15 cells are treated with ETV (FIGS. 9A-9B), the copies of HBV DNA from BC group Is different from that from BS group, the DNA from BC group is higher than that from BS group, and the decline trend of the copies of DNA in BC group is consistent with that of HBV DNA. The above research results mean that the capture method can separate and capture virus particles in cell supernatant. Surprisingly, it is found that HBV DNA in cell supernatant may be mainly derived from NCs particles, while the content of HBV Dane particles is relatively small. The same conclusion is obtained in HepAD38 cell experiment (FIGS. 10A-10B).

### Content of Different HBV Virus Particles in Peripheral Blood

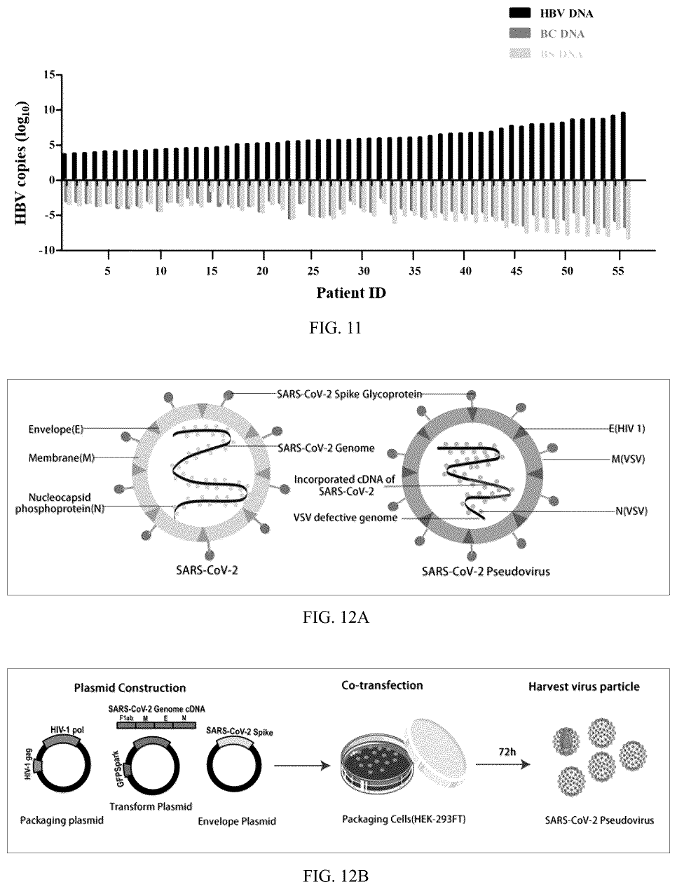

In order to further explore the content of different HBV virus particles in peripheral blood, 56 patients' sera with different titers are randomly selected for detection. As shown in FIG. 11, the results of qPCR show that, except for individual patients, compared with BC group, the DNA content of BS group in serum is higher, especially at high titer serum load, and the content of complete HBV virus particles in serum increased significantly with the increase of HBV DNA copies in serum.

### 3. Detection of Complete Virus Particles in Novel Coronavirus (SARS-CoV-2)

### Embodiment 17

### Materials and Methods

a. Generation and Titration of Pseudovirus

A plasmid containing partial sequences of SARS-CoV-2 ORF1ab gene, N gene, E gene and GFP reporter gene is designed and constructed, and named PLV-SARS-CoV-2-N-GFP. Specifically, HEK-293FT cells are co-transfected with three plasmids: pCMV3-2019-nCoV-Spike(S1+S2), pLV-SARS-CoV-2-N-GFP and pMD2 by lipofectamine 8000 (Beyotime Bio. C0533), and virus supernatant is collected and mixed 48 hours and 72 hours after transfection. After centrifugation at 3,000 g at 4° C. for 10 minutes to remove cell debris, the cell supernatant is placed on 20% sucrose solution and centrifuged at 4° C. at 25,000 revolutions per minute abbreviated as rpm (112,000 g) for 15 hours using a Beckman SW28 rotor (Beckman Coulter, Fullerton, CA, USA) to obtain virus precipitate. The titer of pseudovirus is quantified by HIV-1 Gag p24 DuoSet ELISA kit (Cat: KIT11695, from Sino Biological Inc., China, Beijing).

b. Pseudovirus Identification

HEK-293FT cells are infected by SARS-CoV-2 pseudovirus and control pseudovirus encoding GFP overexpressing hACE2 or empty lentiviral plasmid, the pseudovirus infection is observed under fluorescence microscope at 48 hours and 72 hours after infection, and the supernatant is collected at 72 hours, and the secretion of virus particles is detected by fluorescence quantitative PCR. The efficiency of S protein fusion into pseudovirus is determined by western blotting with mouse anti-SARS-CoV-2 S (S2) monoclonal antibody. The 2019-nCoV nucleic acid detection kit (Sansure Biotech Inc., China) is used to detect the SARS-CoV-2 ORFlab gene in pseudovirus by RT-qPCR to ensure the successfμl integration of SARS-CoV-2 virus genome into lentivirus.

c. Coupling of Carboxy Magnetic Beads with Antibodies

After continuously activating 3 mg carboxy magnetic beads (Fly&Y Bio, Chongqing, China) with 100 μL NHS and 100 μL EDC solution at 25° C. for 30 minutes, the activated MSP-COOH-F1 18030106 is added to the diluted CQ25 antibody (0.6 g/L), and then gently rotated at 4° C. for 4 hours. After separating the supernatant, the complex is gently blocked with 1% BSA solution at 25° C. for 30 minutes. Finally, the coupling effect is evaluated by sodium dodecyl sulfate-polyacrylamide gel electrophoresis (SDS-PAGE) and Coomassie blue staining with the coupled magnetic beads and separated supernatant.

d. Quantitative RT-qPCR Detection of SARS-CoV-2

The antibody-magnetic bead complexes and pseudovirus are mixed in PBS buffer at room temperature for 45 minutes by rotating, and the captured complexes are detected by using a novel coronavirus nucleic acid detection kit to detect level of SARS-CoV-2 RNA in a Bio-Rad CFX96 system. The novel coronavirus nucleic acid detection kit (real-time fluorescence PCR) (Sansure Biotech Inc., China, 001) is used to detect the level of SARS-CoV-2 RNA in the Bio-Rad CFX96 system (Bio-Rad Laboratories, Inc., CA, USA). Each reaction contains 5 μL of sample, 5 μL of sample release agent, 26 μL of 2019-nCoV-PCR reaction solution containing reaction materials, primers and probes, and 4 μL of enzyme mixture containing reverse transcriptase and Taq enzyme. Three replicates of each sample are analyzed and two no-template control (NTC) wells are included to confirm that there is no contamination.

e. Affinity Antibody Screening

After resolution by SDS-PAGE and agarose gel electrophoresis using SARS-CoV-2 pseudovirus lysate and viral particles, respectively, 11 commercially purchased mouse/human anti-SARS-CoV-2 S/M monoclonal antibodies are utilized as the primary antibodies, and the HRP-sheep anti-mouse monoclonal antibodies are used as the secondary antibodies for the screening of optimal specificity and affinity antibodies.

### Western Blotting

30 μL of SARS-CoV-2 S protein overexpressed after transfection of prepared pseudoviruses or 293T cells with wild-type SARS-CoV-2 S glycoprotein vectors is mixed with 6 μL of 6×SDS sample buffer and boiled at 95° C. for 10 minutes. Then, the samples are subjected to SDS-PAGE and western blotting. Mouse anti-S protein antibodies (CQ2, CQ20, CQ25, CQ8, CQ12, CQ001, CQ100, CQ040, CQ042, CQ023 and M1E1) diluted at 1:1000 are used as primary antibodies, and goat anti-mouse IgG (Proteintech Group, Inc., Cat No. SA00003-1) diluted at 1:4000 is used as primary antibody.

### Virus Particle Gel

Denatured samples are electrophoresed in 1×TAE buffer for 2 hours at 70 V on a 1% agarose gel. Virus particles in the gel are transferred to a nylon membrane in 1×TBE buffer according to the siphon principle. After transfer, the membrane is blocked with 5% BSA for 30 minutes, incubated in anti-hiv-1 P24 antibody solution or anti-S antibody solution at 4° C. for 12 hours, and finally washed with 1×TBST buffer and exposed.

f. Particle Size Analysis

The effective particle sizes of carboxy magnetic beads (MB) and magnetic bead-CQ25 antibody complexes (MB-CQ25) are characterized by a NanoBrook 90PLUS PALS particle size analyzer (Brookhaven Instruments Corporation, Holtsville, NY, USA). 2.5 μL sample (25 g/L) is taken and mixed with 3 mL pure water (1‰ triton-100), so that the sample is evenly dispersed in the medium in Brownian motion. If necessary, ultrasound for 15 minutes. The particle sizes are measured after setting parameters according to the instrument instructions, three replicate wells are set at a time and repeated twice. Data are analyzed and plotted using GraphPad Prism 8 (GraphPad Software, Inc., San Diego, CA, USA).

g. Statistical Analysis

SPSS 21.0 for Windows (SPSS, Chicago, IL, USA) statistical software package is used for linear regression, descriptive statistics, repeated measures analysis of variance and two groups of unpaired t test. All significance tests are two-tailed, and p<0.05 is considered statistically significant.

### Results

### (a). Construction and Identification of SARS-CoV-2 Pseudovirus

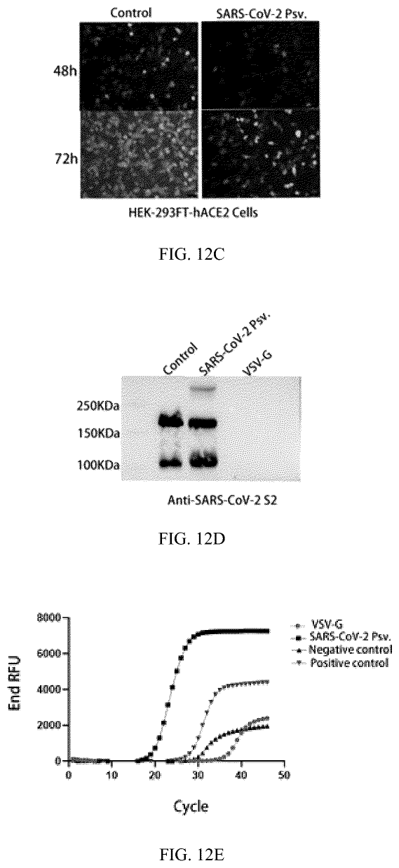

In order to avoid the risk of high pathogenicity and infectivity, the live SARS-CoV-2 virus must be treated under the condition of biosafety level 3, which leads to many research groups being restricted from conducting research related to SARS-CoV-2, even though it may be necessary, very important and urgent. At present, the pseudovirus reported only has S protein or virus nucleic acid, which cannot simulate the complete structure of the virus. Therefore, the inventor constructs a new pseudovirus, which not only expressed spike (S) glycoprotein on the capsid of the virus surface, but also fused some genes of SARS-CoV-2 virus genome, including ORFlab gene, N gene, E gene and GFP coding sequence (FIG. 12A).

In order to construct the SARS-CoV-2 pseudovirus, the inventor first successfully constructed the lentivirus transfer plasmid pLV-SARS-CoV-2-N-GFP and the envelope plasmid pCMV3-2019-nCoV-Spike(S1+S2). SARS-CoV-2 pseudovirus is produced by co-transfection of a three-plasmid system of the transfer plasmid, packaging plasmid and envelope plasmid (i.e., pCMV3-2019-nCoV-Spike(S1+S2), pLV-SARS-CoV-2-N-GFP and pMD2 plasmid) (FIG. 12B), and its infectivity and safety are first tested. HEK-293FT cells overexpressing hACE2 or transduced with empty lentivirus plasmid are infected with SARS-CoV-2 pseudovirus and control VSVG pseudovirus, and the infected cells with green fluorescence are observed under the fluorescence microscope for 48 hours and 72 hours, respectively. At 72 hours after SARS-CoV-2 pseudovirus infection, abundant GFP fluorescence is observed in HEK-293FT-hACE2 cells, indicating that pseudovirus is successfully constructed and transduced (FIG. 12C). In this situation, the cell supernatant is collected at 72 hours after infection and detected by fluorescence quantitative PCR to determine whether there are secreted virus particles in the cell supernatant. There is no positive signal in RT-qPCR results, indicating that virus particles could not replicate and secrete in infected HEK-293FT-hACE2 cells, suggesting that pseudovirus has poor ability to reinfect cells and good biological safety.

The efficiency of S protein fusion into pseudovirus is detected by western blotting with mouse anti-SARS-CoV-2 S (S2) monoclonal antibody. 293T cells are transfected with the vector encoding wild-type SARS-CoV-2 S glycoprotein to express S protein and serve as a positive control. Consistent with the control lane, specific bands can also be found in the lane of SARS-CoV-2 pseudovirus, but no specific bands are found in the corresponding position of VSV-G pseudovirus as negative control. Specifically, two main bands of 190 kDa and 80 kDa respectively correspond to the monomer S protein (S1+S2) and S2 domain (lane 3 in FIG. 12D), with additional Furin sites between S1 and S2 of SARS-CoV-2 protein. The band above 250 kDa may be the product of dimer or trimer S protein.

In order to ensure the successful integration of SARS-CoV-2 virus genome into lentivirus, the inventor uses the 2019-nCoV nucleic acid detection kit (Sansure Biotech Inc., China) to detect SARS-CoV-2 ORFlab gene in pseudovirus by RT-qPCR. As shown in FIG. 12E, compared with the negative sample VSV-G pseudovirus, only SARS-CoV-2 pseudovirus can generate positive detection signals. The above results further confirm that the SARS-CoV-2 pseudovirus is successfully constructed.

### (b). Screening Antibodies for Capturing SARS-CoV-2 Pseudovirus Particles

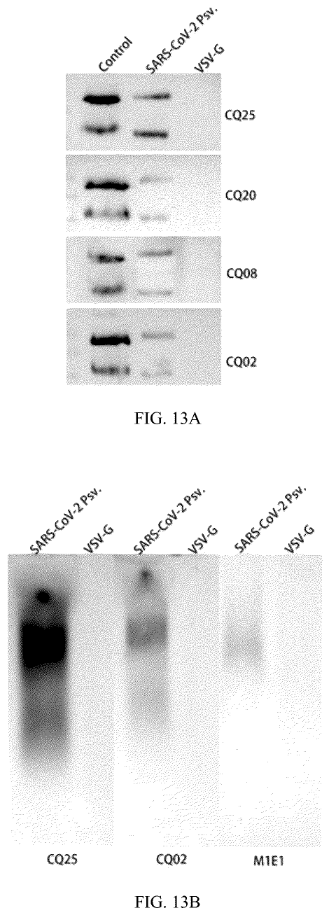

Based on the envelope protein and spike glycoprotein of SARS-CoV-2, the inventor uses 11 monoclonal antibodies, namely CQ02, CQ20, CQ25, CQ08, CQ12, CQ001, CQ100, CQ040, CQ042, CQ023 and M1E1 (purchased from Bioscience (Chongqing) Biotechnology Co., Ltd. and Xiamen Innodx Biotechnology Co., Ltd), the antibodies that could bind to SARS-CoV-2 pseudovirus particles by western blotting and virus particle gel test. In the western blotting experiment, the cleavage products of pseudovirus could be immunoreacted with four antibodies, including CQ20, CQ02, CQ08 and CQ25 (FIG. 13A), due to space steric hindrance, the complete SARS-CoV-2 pseudovirus particles could only be immunoreacted with CQ02, CQ25 and M1E1 antibodies (FIG. 13B). It can be seen that among 11 kinds of antibodies, CQ25 antibody has the highest affinity and specificity for binding with SARS-CoV-2 pseudovirus particles.

### (c). Establishment of a Novel Immune Molecule Detection Method for SARS-CoV-2 Virus Particles

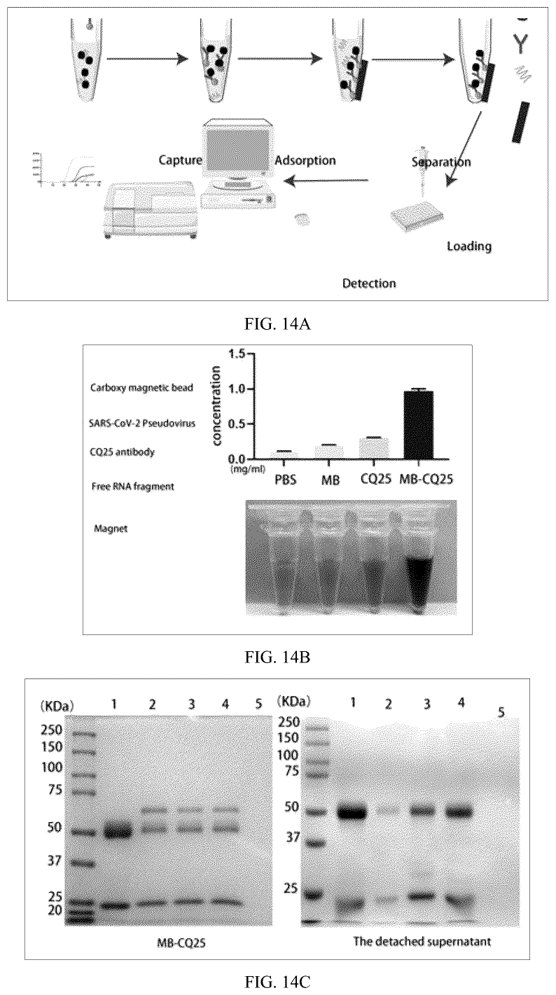

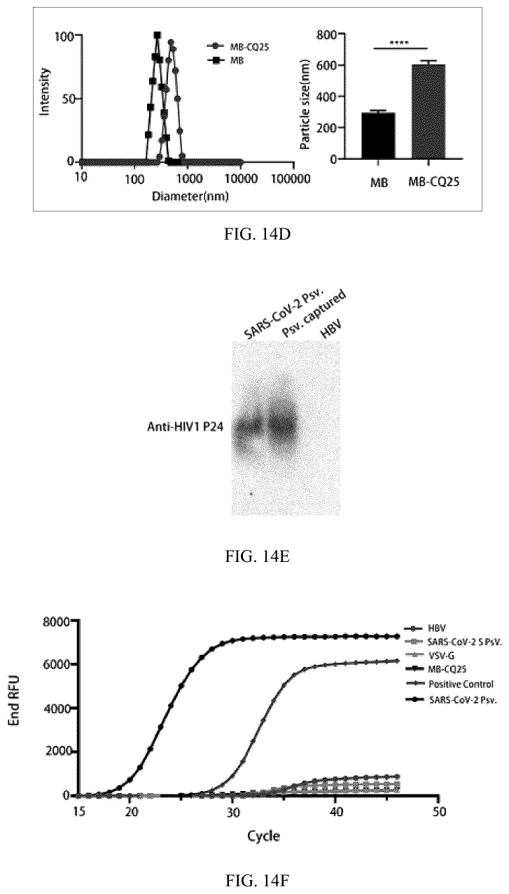

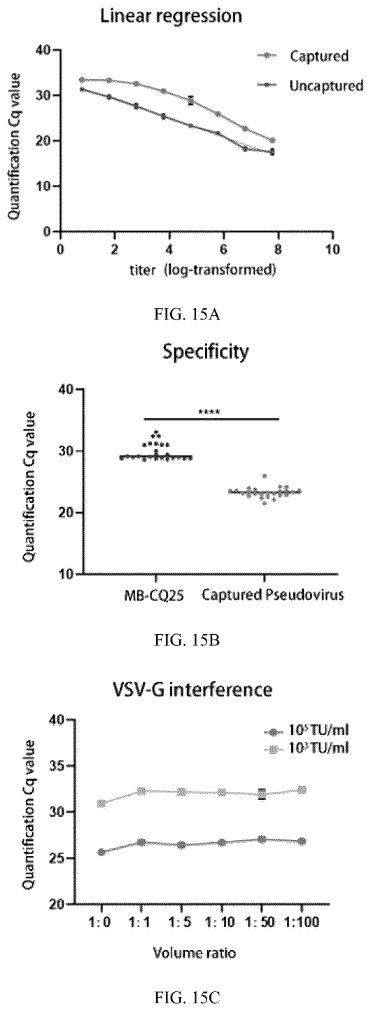

Based on SARS-CoV-2 pseudovirus and screened specific antibody, as shown in FIG. 14A, the inventor designs a brand-new SARS-CoV-2 virus particle detection platform based on the principle of immune molecules. The carboxyl of carboxy magnetic beads is covalently bound to the amino group of the antibody to form a peptide bond, so that the antibody is coupled with the carboxy magnetic beads. After co-incubation with SARS-CoV-2 pseudovirus, the carboxy magnetic beads-CQ25 antibody complex can specifically capture SARS-CoV-2 pseudovirus particles. After the supernatant is separated by the magnetic rack, SARS-CoV-2 pseudovirus particles are enriched and separated from other possible sub-virus particles, such as free RNA fragments, condensate formed by nucleocapsid protein and viral genome RNA, and empty virus particles produced during virus packaging. Finally, the complex is quantitatively and qualitatively detected by fluorescence quantitative PCR. The combination of immune and molecule principles further ensures that the detection platform only targets complete virus particles.

In order to establish an immune molecule detection platform for complete SARS-CoV-2 virus particles, the inventor first successfully couples carboxy magnetic beads and CQ25 antibody. The protein concentration of the complex after coupling is measured by BCA method to evaluate the coupling effect (FIG. 14B). Compared with the untreated carboxy magnetic beads, the complex has a higher absorbance at 562 nanometers (nm), indicating that the antibody has been successfully coupled with the magnetic beads. In SDS-PAGE results, the coupled magnetic beads all have specific bands at the corresponding positions of the antibody, while the untreated carboxy magnetic beads have no specific bands, which further proves that the magnetic beads are successfully coupled with the antibody. With the increasing amount of added antibody, the antibody in the detached supernatant increased continuously after the coupling is completed (FIG. 14C), which indicates that 3.3 mg of carboxy magnetic beads could be completely coupled with at least 60 μg of antibody. In addition, the results of particle size analysis show that the average effective diameter of the complex after coupling is about 600 nm and carboxy magnetic beads are about 300 nm, and the significant increase of particle size further proves that the coupling between antibody and the carboxy magnetic beads is effective (FIG. 14D).

In the disclosure, whether the SARS-CoV-2 pseudovirus can be captured by carboxy magnetic beads coupled with specific antibodies is tested. Because HIV1 P24 is the most abundant marker protein in lentivirus capsid, the inventor uses mouse anti-HIV 1 p24 monoclonal antibody to carry out virus particle gel experiment, and then the virus capsid protein of the captured complete virus particle is successfully identified (FIG. 14E). Then nonspecific samples such as SARS-CoV-2 S pseudovirus, VSV-G pseudovirus and HBV virus are used to further verify the specificity of virus capture. The captured magnetic bead-antibody-virus complex is detected by RT-qPCR. Compared with the negative sample, only the SARS-CoV-2 pseudovirus constructed by the inventor could detect the positive signal, which indicates that the portable platform could capture the complete SARS-CoV-2 pseudovirus particles with good specificity (FIG. 14F).

### (d). Verification of the Complete SARS-CoV-2 Virus Particle Detection Platform Based on Immune Molecules

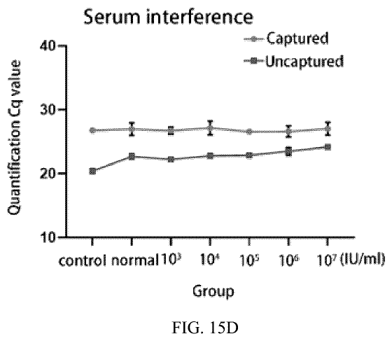

The live SARS-CoV-2 virus has dangerous pathogenicity and infectivity and must be treated under biosafety level 3 conditions, and therefore, the constructed SARS-CoV-2 pseudovirus has to be used to verify the complete virus particle detection platform. Linear range means that there is a direct correlation between signal and substance concentration within a certain range. The titer of SARS-CoV-2 pseudovirus detected by P24 ELASE kit is about 6.07×10⁷ TU/mL. Due to the lack of standards, the original SARS-CoV-2 pseudovirus is diluted by 10 times gradient, and fluorescence quantitative PCR is carried out to determine the linear range. When the titer of pseudovirus is in the range of 10²-10⁷ TU/mL, the quantification Cq value of the immune molecule detection method has a linear relationship with its titer (log transformed) y−2.57x+40.203 (R²=0.99, FIG. 15A), while the linear range of direct qPCR is in the range of 10-10⁷ TU/mL, y=2.070x+33.23 (R²=0.98). These two different detection methods produce different signals for the same drop of virus, which is probably the combined result of the differences in methods and the interference of incomplete virus particles, but the ratio of complete virus particles to incomplete virus particles produced during viral packaging needs to be further investigated.

As shown in FIG. 15B, there is a significant difference between 24 negative samples, i.e., carboxy magnetic bead-antibody (CQ25-MB) and 24 positive samples after capturing pseudovirus P<0.0001, ****. When the average titer +1.96 standard deviation (SD) of the negative samples is used as the limit of detection (LOD), the LOD is 10³ TU/mL. Although the quantification Cq value can be measured when the titer of pseudovirus is lower than 10³ TU/mL, the result of fluorescence quantitative PCR is still considered negative.

The inventor uses VSV-G pseudovirus and sera with different copies of HBV to verify the anti-interference ability of this method. As shown in FIG. 15C, adding different volumes of VSV-G pseudovirus has little effect on the quantitative and qualitative detection of complete SARS-CoV-2 pseudovirus particles, and the coefficients of variation between analyses are 1.85% and 1.69% respectively. Normal human serum contains a lot of albumin and various antibodies, it is necessary to consider whether these factors will affect the specificity and stability of the detection. Therefore, the inventor compares the interference ability of normal human serum and serum of patients with different copies of HBV on direct qPCR and immune molecule detection, respectively (FIG. 15D). Surprisingly, the anti-interference ability of the immune molecule detection of the disclosure to serum and HBV is more significant than that of direct qPCR detection, and the coefficient of variation of intra-assay analysis is 0.83% and 5.19%, respectively. It is suggested that the novel immune molecule detection method of the disclosure has good specificity and stability for detecting complete SARS-CoV-2 particles.

What is claimed is:

1. An immune molecule virus particle detection kit, comprising a monoclonal antibody, biotin, magnetic beads and streptavidin; wherein the monoclonal antibody is a monoclonal antibody of a virus envelope antigen; and
wherein the immune molecule virus particle detection kit is configured to detect virus particles by: modifying the monoclonal antibody of the virus envelope antigen with the biotin to obtain a biotin-modified monoclonal antibody, and coupling the magnetic beads with the streptavidin to obtain streptavidin-coupled magnetic beads; incubating the biotin-modified monoclonal antibody with a virus-containing solution to form a complex with the virus particles or the antigen; then, adding the streptavidin-coupled magnetic beads for incubation to make the streptavidin on the magnetic beads be combined with the biotin-modified monoclonal antibody to capture the virus particles with envelopes; separating a supernatant of the captured virus particles by a magnetic separator to make complete virus particles, empty-shell viruses and free envelope antigens be separated from other virus components to thereby obtain magnetic bead conjugates; and detecting the magnetic bead conjugates qualitatively or quantitatively by polymerase chain reaction (PCR) amplification.

2. The kit as claimed in claim 1, wherein the virus envelope antigen is a host receptor-binding viral protein; the other virus components are sub-virus particle components comprising a protein-virus ribonucleic acid (RNA) or deoxyribonucleic acid (DNA) complex or a free virus gene fragment; and the PCR amplification is one of fluorescence quantitative PCR and digital PCR isothermal amplification.

3. The kit as claimed in claim 2, wherein the biotin-modified monoclonal antibody is obtained by dialyzing the monoclonal antibody with a sodium bicarbonate buffer with a power of hydrogen (pH) value of 8.0 or a boric acid buffer with a value of pH 8.6 to obtain a monoclonal antibody solution, adding biotin dissolved in dimethyl sulfoxide (DMSO) into the monoclonal antibody solution, continuously stirring at room temperature, and keeping the temperature for 2-4 hours (h); adding ammonium chloride (NH₄Cl), and stirring at room temperature for 5-15 minutes (min); removing free biotin to obtain a loading sample; loading the loading sample on a molecular sieve column, eluting with phosphate buffered saline (PBS), and collecting proteins; adding sodium azide and bovine serum albumin (BSA) to form the biotin-modified monoclonal antibody as a product to be combined;
wherein the streptavidin-coupled magnetic beads is obtained by taking the magnetic beads into an Eppendorf (EP) tube, performing magnetic separation on the magnetic beads, and washing with a precooled 4-morpholinoethanesulfonic acid (MES) buffer; applying a magnetic field to remove a supernatant, adding an N-hydroxysuccinimide (NHS) solution and a 1-Ethyl-3-[3-dimethylaminopropyl]carbodiimide hydrochloride (EDC) solution with a same amount as the NHS solution into the EP tube, shaking, and activating the magnetic beads at 20-30 Celsius degree (° C.) for 20-40 min; washing the activated magnetic beads with the precooled MES buffer with a magnetic rack; diluting streptavidin to be coupled with the precooled MES buffer to obtain a diluted streptavidin suspension, resuspending the activated magnetic beads after washing with the precooled MES buffer, and shaking to disperse all the magnetic beads, thereby to obtain an activated magnetic bead suspension; taking and adding the activated magnetic bead suspension into the diluted streptavidin suspension, and rotating and mixing uniformly at 4° C. for 4 h; applying the magnetic field to removing a supernatant, adding a BSA blocking solution into the EP tube, and rotating at 20-30° C. for 20-40 min; washing the magnetic beads after coupling by the magnetic rack with the PBS; transferring a preservation solution into the EP tube, suspending the magnetic beads coupled with the streptavidin, and preserving at 4° C., so as to obtain the streptavidin-coupled magnetic beads; and
wherein the virus particles bound to the biotin-modified monoclonal antibody and captured by the streptavidin-coupled magnetic beads are performed by: taking a cell supernatant into another EP tube, adding the biotin-modified monoclonal antibody for incubation and binding, and rotating at 20-30° C. for 5-15 min; adding the streptavidin-coupled magnetic beads, uniformly mixing, and rotating and binding at 20-30° C. for 30-50 min; and discarding the supernatant after magnetic field is applied to obtain the complete virus particles, the empty-shell viruses and the free envelope antigens.

4. The kit accord to claim 1, wherein the virus particles are complete virus particles selected from the group consisting of hepatitis A virus (HAV), hepatitis B virus (HBV), hepatitis C virus (HCV), hepatitis D virus (HDV), hepatitis E virus (HEV), novel coronavirus (SARS-CoV-2), human immunodeficiency virus (HIV), influenza virus, Partial pulmonary virus, human papillomavirus (HPV), herpes virus, herpesvirus hominis, Zika virus, Ebola virus (EBV), human T-lymphocytic virus, avian influenza virus, hog cholera virus (CSFV), poliovirus, rabies virus, adenovirus, and lentivirus.

5. A method for detecting immune capture molecules of complete HBV virus particles, comprising antibody-magnetic bead coupling, HBV virus particle capture and real-time fluorescence quantitative PCR;
wherein the antibody-magnetic bead coupling comprises:
mixing carboxy magnetic beads, an NHS solution and an EDC with a same amount as the NHS solution in a buffer to activate magnetic beads, and mixing and reacting the activated magnetic beads with an antibody to be coupled in a coupling buffer to obtain an antibody-magnetic bead coupling reaction product; and
wherein the antibody to be coupled is at least one of a PreS1 antibody and an HBc antibody.

6. The method as claimed in claim 5, wherein the antibody-magnetic bead coupling specifically comprises:
taking the carboxy magnetic beads into an EP tube, performing magnetic separation on the carboxy magnetic beads, and washing with an IVIES buffer for three times; applying a magnetic field to remove a supernatant, quickly adding the NHS solution and the EDC solution with the same amount of the NHS solution into the EP tube, shaking vigorously, and continuously activating the carboxy magnetic beads at 25° C. for 30 min; washing the activated carboxy magnetic beads with the MES buffer with a magnetic rack for three times; diluting the antibody to be coupled with the IVIES buffer until a final antibody concentration is 0.6 grams per liter (g/L) to obtain a diluted antibody suspension; resuspending the activated carboxy magnetic beads with the IVIES buffer and shaking vigorously to ensure that the activated carboxy magnetic beads are completely dispersed to thereby obtain an activated magnetic bead suspension; taking and adding the activated magnetic bead suspension into the diluted antibody suspension for 5 times, mixing immediately after adding the activated magnetic bead suspension each time, rotating and mixing evenly at 4° C. for 4 h; preparing 5% of BSA blocking solution; applying the magnetic field to remove a supernatant, quickly adding the BSA blocking solution into the EP tube, and rotating at 25° C. for 30 min; washing the carboxy magnetic beads after coupling by the magnetic rack with PBS for three times; transferring a preservation solution to the EP tube, suspending the carboxy magnetic beads, and preserving at 4° C., so as to obtain an antibody-coupled magnetic bead preservation solution.

7. The method as claimed in claim 6, wherein the HBV particle capture comprises:
taking the antibody-coupled magnetic bead preservation solution, performing magnetic separation to discard a supernatant of the antibody-coupled magnetic bead preservation solution, and then washing twice with the PBS; taking a cell supernatant or a serum of a hepatitis B patient into another EP tube and diluting the cell supernatant or the serum of the hepatitis B patient with the PBS to obtain a diluted sample; adding an antibody-magnetic bead conjugate from the washed antibody-coupled magnetic bead preservation solution to the diluted sample, mixing evenly, and rotating at 25° C. for 40 min to capture the complete HBV virus particles, so as to obtain a captured complex.

8. The method as claimed in claim 7, wherein the real-time fluorescence quantitative PCR comprises:
resuspending the captured complex with 50 microliters (μL) PBS to obtain a captured complex suspension, transferring the captured complex suspension to 8-strip PCR tubes, and removing a supernatant of the 8-strip PCR tubes with the magnetic rack of a PCR plate; placing required reagents at room temperature in advance to avoid light, setting standards A-D, a negative control and a positive control, adding 5 μL sample release agent to each well of the 8-strip PCR tubes, instantly centrifuging, beating and mixing evenly, and standing in the dark for min; preparing a PCR mixed solution per person comprising 38 μL reaction solution+2 μL enzyme mixed solution+0.2 μL internal standard; and performing the real-time fluorescence quantitative PCR for cyclic amplification detection according to the following procedures:
uracil N-glycosylase (UNG) reaction at 50° C. for 2 min, one cycle; Taq enzyme activation at 94° C. for 5 min, one cycle; denaturation at 94° C. for 15 seconds (s), 45 cycles; annealing, extension and fluorescence collection at 57° C. for 30 s, 45 cycles; and instrument cooling at 25° C. for 10 s, one cycle.

9. A method for detecting complete virus particles of SARS-CoV-2, comprising:
pseudovirus generation and titration, pseudovirus identification, affinity antibody screening, carboxy magnetic beads and antibody coupling, SARS-CoV-2 quantitative RT-qPCR detection, western blotting, virus particle gel and particle size analysis;
wherein the pseudovirus generation and titration comprises:
co-transfecting HEK-293FT cells with a pCMV3-2019-nCoV-Spike(S1+S2) plasmid, a pLV-SARS-CoV-2-N-GFP plasmid and a pMD2 plasmid by Lipofectamine™ 8000, collecting a virus supernatant after co-transfection and mixing, centrifuging the virus supernatant to remove cell debris, placing a cell supernatant after the centrifuging on a sucrose solution, and obtaining a virus precipitate by centrifugation with a Beckman SW28 rotor; and
quantifying a titer of the pseudovirus from the virus precipitate by using an HIV-1 Gag p24 DuoSet ELISA kit.

10. The method as claimed in claim 9, wherein the pseudovirus is generated specifically by co-transfecting the HEK-293FT cells with the pCMV3-2019-nCoV-Spike(S1+S2) plasmid, the pLV-SARS-CoV-2-N-GFP plasmid and the pMD2 plasmid with the Lipofectamine™ 8000, collecting the virus supernatant at 48 h and 72 h after co-transfection and mixing, centrifuging the virus supernatant at 3000 g at 4° C. for 10 min to remove the cell debris, placing the cell supernatant on 20% sucrose solution, and obtaining the virus precipitate containing a SARS-CoV-2 pseudovirus by centrifugation with the Beckman SW28 rotor at 112,000 g at 4° C. for 15 h;
wherein the pseudovirus identification comprises:
performing pseudovirus infection in vitro by transfecting HEK-293FT cells overexpressing human angiotensin-converting enzyme 2 (hACE2) or transduced with empty lentivirus plasmid with the SARS-CoV-2 pseudovirus and a control pseudovirus encoding green fluorescent protein (GFP) into a 48-well plate for 48 h and 72 h, observing the pseudovirus infection under a fluorescence microscope and collecting a supernatant at 72 h, and detecting secretion of virus particles by a fluorescence quantitative PCR;
wherein the affinity antibody screening comprises:
performing sodium dodecyl sulfate-polyacrylamide gel electrophoresis (SDS-PAGE) and agarose gel electrophoresis respectively on a SARS-CoV-2 pseudovirus lysate and the virus particles for resolution and membrane transfer, using mouse/human anti-SARS-CoV-2 S/M monoclonal antibodies as primary antibodies and horseradish peroxidase (HRP)-sheep anti-mouse monoclonal antibodies as secondary antibodies to screen an antibody with optimal specificity and affinity; and
wherein the carboxy magnetic beads and antibody coupling comprises:
activating the carboxy magnetic beads continuously with an NHS solution and an EDC solution at 25° C. for 30 min to obtain activated carboxy magnetic beads MSP-COOH-F1;
adding the activated carboxy magnetic beads MSP-COOH-F1 to a diluted antibody CQ25, mixing and rotating at 4° C. for 4 h, separating a supernatant to obtain an antibody-coupled magnetic bead complex, blocking the antibody-coupled magnetic bead complex with 1% BSA solution at 25° C. for 30 min; evaluating a coupling effect by the SDS-PAGE and Coomassie blue staining with the antibody-coupled magnetic bead complex and the separated supernatant.

11. The method as claimed in claim 10, wherein the SARS-CoV-2 quantitative RT-qPCR detection comprises:
mixing the antibody-coupled magnetic bead complex and the SARS-CoV-2 pseudovirus in a PBS buffer at room temperature for 45 min to obtain a captured complex, and detecting a SARS-CoV-2 RNA level of the captured complex by using a novel coronavirus nucleic acid detection kit in a Bio-Rad CFX96 system.
